(19) United States
(12) Patent Application Publication
(10) Pub. No.: US 2008/0187944 A1
(43) Pub. Date: Aug. 7, 2008

(54) Butyrylcholinesterase as a marker of low-grade systemic inflammation

(76) Inventors: Appa Rao Allam, Visakhapatnam (IN); Sridhar Gumpeny, Visakhapatnam (IN); Undurti Das, Shaker Heights, OH (US)

Correspondence Address: Undurti N. Das;#321, 13800 Fairhill Road, Shaker Heights, OH, 44120, US

(21) Appl. No.: 12/011,451

(22) Filed: Jan. 28, 2008

Related U.S. Application Data
(60) Provisional application No. 60/898,450, filed on Jan. 31, 2007.

(51) Int. Cl. C12Q 1/46

(52) U.S. Cl. 435/20

(57) ABSTRACT

The present invention relates to a strategy or method of detecting, diagnosing, and prognosticating low-grade systemic inflammatory conditions such as insulin resistance, type 2 diabetes mellitus, hypertension, hyperlipidemias, and Alzheimer's disease; various cancers, and acute and chronic collagen vascular diseases including rheumatoid arthritis (RA), and lupus; proliferative diabetic retinopathy; macular degeneration; multiple sclerosis, psoriasis, chronic renal failure, end-stage renal disease, glomerulonephritis including minimal change nephropathy, proliferative glomerulonephritis, nephritis secondary to underlying systemic diseases using plasma and/or tissue levels of acetylcholinesterase and butyrylcholinesterase as a marker. The method employed to detect acetylcholinesterase and butyrylcholinesterase could include various methods such as but not limited to turbimetric test, or ELISA (enzyme linked immunosorbent assay), fluorometric, radioimmunoassay, and spectrophotometric methods.

### FIELD OF INVENTION

The invention generally relates to a strategy or method of detecting and diagnosing low-grade systemic inflammatory conditions such as insulin resistance, type 2 diabetes mellitus, hypertension, hyperlipidemias, and Alzheimer's disease using plasma and/or tissue levels of butyrylcholinesterase as a marker. In addition, for the prevention, treatment, and monitor prognosis of various cancers, and acute and chronic inflammatory conditions such as rheumatoid arthritis (RA), systemic lupus erythematosus (SLE), progressive systemic sclerosis (PSS), mixed connective tissue disorder (MCTD), vasculitis; and other disorders caused by uncontrolled angiogenic activity such as proliferative diabetic retinopathy; other eye disorders such as macular degeneration; and central nervous system disorders such as multiple sclerosis, Alzheimer's disease; skin problems such as psoriasis, renal conditions such as chronic renal failure, end-stage renal disease, glomerulonephritis such as minimal change nephropathy, various forms of proliferative glomerulonephritis, nephritis secondary to underlying systemic diseases such as collagen vascular diseases, lymphoma and leukemias; and other disorders in which cell proliferation and angiogenesis plays a dominant role and which are also characterized by low-grade systemic inflammation, development of a reliable, unique and specific marker is essential. The present invention relates to a strategy or method of detecting, diagnosing, and prognosticating all the above-mentioned conditions using plasma and/or tissue levels of butyrylcholinesterase as a marker.

### BACKGROUND OF THE INVENTION

Inflammation

Inflammation is a complex reaction to injurious agents, either external or internal, that consists of both vascular and cellular responses. Inflammation may be local or systemic, and it can be acute or chronic. During the inflammatory process, the reaction of blood vessels is unique that leads to the accumulation of fluid and leukocytes in extravascular tissues. The reaction of blood vessels can be in the form of vasodilatation, that is seen in the form of hyperemia at the site(s) of injury, that does the essential function of increasing the blood supply to the injured tissue/organ so that adequate elimination of the inflammation-inducing agent is achieved and/or repair process can occur after the inflammation subsides. Thus, both injury and repair are two faces of the inflammatory process that are very closely intertwined such that it is difficult to separate these two processes. In fact, in majority of the instances, both inflammation to injury and repair occur almost simultaneously.

Inflammation is fundamentally a protective response whose ultimate goal is to eliminate the injury-inducing agent (that could be a microorganism, physical stimuli, chemical agent, etc.), prevent tissue damage and/or initiate the repair process. Without inflammation there is no life since, in the absence of adequate inflammation cell/tissue injury would go unchecked, the damage done to the cells/tissues/organs would never heal, and ultimately this may lead to the death of the organism itself. Thus, inflammation is both beneficial and potentially harmful.

It is now believed that low-grade systemic inflammation plays a significant role in the pathogenesis of conditions such as insulin resistance, type 2 diabetes mellitus, hypertension, hyperlipidemias, Alzheimer's disease, proliferative diabetic retinopathy; other eye disorders such as macular degeneration, and minimal change nephropathy. In addition, inflammatory events both acute and chronic are at the centre of conditions such as rheumatoid arthritis (RA), systemic lupus erythematosus (SLE), progressive systemic sclerosis (PSS), mixed connective tissue disorder (MCTD), vasculitis; psoriasis, glomerulonephritis of different etiologies including but not limited to proliferative glomerulonephritis, nephritis secondary to underlying systemic diseases such as collagen vascular diseases, lymphoma and leukemias, and central nervous system disorders such as multiple sclerosis. In all these acute, chronic and low-grade systemic inflammatory conditions uncontrolled angiogenic activity also occurs that either precedes or closely follows cell proliferation (for example as seen in proliferative glomerulonephritis and diabetic retinopathy). Thus, development of a reliable, unique and specific marker to detect acute, chronic and low-grade systemic inflammation is essential.

In order to develop newer diagnostic tools, it is important to understand pathophysiological mechanisms of inflammation that has been enumerated briefly below.

### COMPONENTS OF INFLAMMATORY RESPONSE

The inflammatory response mainly consists of two components: a vascular response and a cellular response, both of which are integral and essential parts of the inflammatory reaction. The vascular and cellular reactions of both acute and chronic inflammation are mediated by chemical factors (that could be proteins, lipids, or lipoproteins in nature) that are secreted by various cells that take part in the inflammatory process either directly and/or responding to the inflammatory stimulus. These chemical mediators of the inflammation acting singly, in combinations, or in sequence, amplify the tissues/organs response to the stimulus and influence the course of inflammation. In addition, necrosis or apoptosis of the cells or tissues that occur during the process of inflammation/repair themselves elaborate or liberate certain chemicals that also take part in inflammation. Once the inflammatory process is initiated, tissues/organs try to elaborate certain anti-inflammatory chemicals and signals that try to minimize tissue damage and eliminate the harmful effects of inflammation. Thus, ultimately recovery of a tissue/organ from the inflammatory process and regaining of its function depends to a large extent on the balance between pro- and anti-inflammatory chemicals and events that occur as a result of these mutually antagonistic processes. Once inflammation is terminated either by endogenous mediators/repair processes and/or by modern medical techniques (that may include antibiotics, anti-inflammatory drugs, chemical and surgical measures) and the offending agent is successfully removed, all the secreted mediators and the cellular responses are either broken down or dissipated and the tissues/organs in question revert to their natural physiological state, to the extent possible, depending on the degree of damage and repair that has occurred.

Since several circulating cells and chemical mediators participate in both acute and chronic inflammation, it is possible to measure either the expression of certain molecules on the surface of these circulating cells, chemicals that are released by these circulating cells or both as markers of inflammation. In majority of the instances, especially when the inflammatory process is on the surface of the body and is in an acute form, as evidenced by rubor, tumor, calor, dolor, and functiolaesa (redness, swelling, heat, pain, and loss of function respectively) perhaps, no specific tests are necessary to measure the presence or absence of inflammation. But, specific tests become necessary when certain chronic inflammatory processes need to be detected that are taking place deep inside the body or in certain internal organs that are not very obvious on clinical examination. This is especially so since, at present, it is believed that many chronic diseases that have hitherto been thought to be degenerative processes or due to ageing, seem to be due to low-grade systemic inflammation. Thus, obesity, hyperlipidemia, essential hypertension, type 2 diabetes mellitus, coronary heart disease (CHD), and metabolic syndrome X (that is characterized by abdominal obesity, hypertension, hyperlipidemia, and insulin resistance) are now thought to be diseases of low-grade systemic inflammation [1] and hence, several studies are examining the possibility of utilizing certain markers of inflammation either to predict the development of these diseases and/or prognosticate their course.

Components of Acute Inflammation

Acute inflammation that is a rapid response to an injurious agent has mainly three components: a) alterations in the diameter of the blood vessels generally vasodilatation whose main purpose is to increase blood flow to the site of inflammation; b) structural changes in the microvasculature such that it permits plasma proteins and leukocytes to leave the circulation to aid in the pathobiology of inflammation both in injury and repair processes; c) accumulation of leukocytes at the site of inflammation and their activation to release chemical mediators of inflammation and to eliminate the offending organism or agent. Various agents that generally trigger acute inflammation include: infections by bacterial, viral, fungi, and parasitic organisms and their toxins; trauma; physical and chemical agents such as burns, radiation, and environmental or man made chemicals; foreign bodies such as splinters, thorns, sutures; abnormal immune reactions especially hypersensitivity reactions. Although inflammation induced by these various agents could have some distinct features, in general, all inflammatory reactions share same basic features.

Vascular Changes

i. Vasodilatation: This is one of the most essential components of inflammation. Vasodilatation is an early and important manifestation of acute inflammation. Sometimes, early vasodilatation is followed by transient vasoconstriction. The main purpose of vasodilatation is to increase blood flow to the site of inflammation to carry circulating proteins and other mediators to aid inflammation. Initially, the existing blood vessels undergo dilatation but at a later stage depending on the demand and necessity and the mediators that are released at the site of inflammation newer capillary beds are opened. Vasodilatation is followed by increased permeability of the microvasculature that ultimately allows outpouring of protein rich fluid and extravasation of leukocytes to the site(s) of inflammation. Prior to the extravasation of leukocytes, as a result of leakage of protein and vasodilatation there could occur stasis of blood flow reflected by an increase in the concentration of red blood cells in the smaller vessels resulting in increased viscosity of the blood. As a result of this stasis, leukocytes, especially polymorphonuclear leukocytes (PMNs) accumulate along the vascular endothelium and over a period of time escape from the blood vessels into the interstitial tissue.

The exact mechanism(s) and the mediators involved in vasodilatation process during inflammation are still not known. Recent studies showed that nitric oxide (NO) produced by endothelial cells and possibly other cells seem to have a pivotal role in vasodilatation of inflammation. NO is a potent vasodilator and platelet anti-aggregator and its local production could indeed be one of the important mediators of vasodilatation seen during inflammation. Several other mediators of vasodilatation may include carbon monoxide (CO), prostaglandins (PGs) including other eicosanoids, bradykinin and other kinins, and histamine. The final degree of vasodilatation at a given site of inflammation could depend on the amount of each of these possible mediators released from various cells, the balance between vasodilator and vasoconstrictor mediators released and their respective inactivators. These various mediators are released by macrophages, monocytes, infiltrating leukocytes, lymphocytes, endothelial cells, and other cells present at the site of inflammation. Furthermore, there appears to be a close interaction between these various vasoactive molecules. For instance, it was observed that myeloperoxidase (MPO) released by activated PMNs not only generates cytotoxic oxidants but also impacts deleteriously on NO-dependent signaling cascades and thus could influence vasodilatation during inflammation. MPO increased tyrosine phosphorylation and p38 mitogen-activated protein kinase activation; MPO-treated PMNs released increased amounts of free radicals, and enhanced PMN degranulation [2]. MPO, a highly abundant, PMN-derived heme protein facilitates oxidative NO consumption and impairs vascular function in animal models of acute inflammation [3]. Superoxide anion (O₂.⁻), produced by PMNs during acute inflammation has the ability to inactivate NO and thus, reduce its half-life and activity. Thus, there appears to be a close interaction between various mediators of acute inflammation and this may have relevance to the pathogenesis of inflammation including vasodilatation seen during this process.

ii. Vascular leakage: Leakage of circulating protein into the extravascular tissue results in edema that is one of the hallmarks of inflammation. This leakage of proteinaceous fluid is due to the formation of endothelial gaps in venules, direct endothelial damage, necrosis or detachment, leukocyte-mediated endothelial injury that ultimately results in the loss of circulating protein into the extravascular tissue [4].

Although, exact details as to the chemical mediators and the sequence of their production is not clear, it is suffice for the present discussion to know that cytokines such as interleukin-1 (IL-1), tumor necrosis factor-α (TNF-α), interferon-γ (IFN-γ), vascular endothelial growth factor (VEGF), histamine, substance P, free radicals, nitric oxide and other yet unidentified chemicals seem to play a significant role in vasodilatation, vascular leakage, and diapedesis of leukocytes [5]. On the other hand, PMN-induced damage to vascular endothelial cells is believed to be due to increased production of reactive oxygen species (ROS), inducible nitric oxide (iNO) and its metabolites (such as OCl.), ozone, and release of cytokines. The main purpose of ROS, iNO, and ozone appears to be to kill and eliminate the invading microorganisms. In view of their ability to diffuse across cell membranes and tissues and potent actions, they produce collateral damage to the surrounding cells/tissues. In addition to their pro-inflammatory actions, ROS, iNO, IL-1, TNF, and IFN and to some extent VEGF also have modulatory influence on vascular reactivity, endothelial cell function, smooth muscle cell proliferation, expression of adhesion molecules, leukocyte function, and extracellular matrix production. These actions ultimately influence the inflammatory process, repair of the inflamed tissues/organs, and functional integrity of the target tissues/organs. The therapeutic application of the knowledge gained from the fundamental understanding of inflammation and its various molecular events led to the development of various monoclonal antibodies that neutralize the actions of IL-1, TNF-α, IFN, and VEGF. For example, it is now known that age-related macular degeneration (AMD) is due to increased production of VEGF in the retinal tissue. Recent studies showed that anti-VEGF therapies are of significant benefit in AMD [6]. On the other hand, monoclonal antibodies against IL-1, and TNF-α failed to show any significant benefit in acute systemic inflammatory condition such as sepsis and septic shock [7] suggesting that our understanding of inflammation is still inadequate to develop therapeutically meaningful approaches. In this context, the role of free radicals in vascular reactivity during inflammation may prove to be interesting. Free radicals such as hydrogen peroxide (H₂O₂), O₂.⁻, NO, nitrated lipids etc., have vasoactive actions. NO is a vasodilator, whereas O₂.⁻ and other free radicals have vasoconstrictor actions. In fact, it is believed that O₂.⁻ could be the vasoconstrictor that produces coronary vasospasm leading acute angina. In view of the contrasting actions of NO and O₂.⁻ on vascular reactivity, the final diameter of the blood vessels may depend on the balance between NO and O₂.⁻ produced at the site of inflammation. Since tissue antioxidant defenses such as superoxide dismutase (SOD), catalase, and glutathione try to neutralize, suppress, or antagonize the actions of free radicals, the tissue destructive properties and vasoconstrictor actions of free radicals are determined to a large extent on the tissue concentrations of these antioxidants. Furthermore, NO by itself can neutralize the actions of O₂.⁻ and hence the balance between these two molecules could be yet another modulator of inflammation.

b) Cellular Events

i. Leukocyte extravasation and chemotaxis: In order to eliminate the inciting agent responsible for inflammation and initiate the repair process, it is critical that leukocytes are delivered to the site of injury. One of the major functions of leukocytes is to ingest the offending agent, kill bacteria and other microbial organisms, and remove the necrotic tissue, debris and foreign material. In the process of performing these important functions, leukocytes also induce tissue damage and in some instances, may prolong inflammation. Leukocytes need to extravagate from inside the blood vessels in order to bring about these actions. For this purpose, leukocytes adhere to the endothelial lining of the blood vessels, transmigrate across the endothelium (a process called as diapedesis), and migrate in interstitial tissues toward the chemotactic stimulus and reach the site of inflammation or injury [8]. For this extravasation to occur and for the leukocytes to adhere and transmigrate from the blood into tissues, both leukocytes and endothelial cells express complementary adhesion molecules, whose expression, in turn, is regulated largely by cytokines. The adhesion receptors involved in this process belong to are four major molecular families, namely: selecting, immunoglobulin superfamily, integrins, and mucin-like glycoproteins. The multi-step process of leukocyte migration through blood vessels involves: leukocyte rolling, activation and adhesion of leukocytes to endothelium, transmigration of leukocytes across the endothelium, piercing the basement membrane, and finally migration towards chemoattractants emanating from the site of injury or inflammation. Although almost all molecules may have a role in several of these processes, certain molecules play a dominant role in certain processes. For instance, selectins play a major role in rolling; chemokines in activating the neutrophils to increase avidity of integrins; integrins in firm adhesion; and CD31 (PECAM-1) in transmigration [9].

The induction of adhesion molecules on endothelial cells may occur by a number of mechanisms. For example, histamine, thrombin, and platelet activating factor (PAF) stimulate the redistribution of P-selectin from its intracellular stores to the cell surface; whereas macrophages, mast cells, and endothelial cells secrete pro-inflammatory cytokines such as IL-1, TNF-α, and chemokines that act on endothelial cells and induce the expression of several adhesion molecules. This results in the expression of E-selectin on the surface of endothelial cells. Simultaneously, leukocytes express carbohydrate ligands for the selectins that allow them to bind to the endothelial selectins [10]. This binding of leukocytes to endothelium is a low-affinity interaction that is easily disrupted by the flow of blood. This alternate process of binding, disruption of the binding, and binding once again of leukocytes to endothelial cells results in rolling of leukocytes on the surface of endothelium.

On the other hand, IL-1 and TNF-α and possibly other such pro-inflammatory cytokines induce the expression of ligands for integrins such as VCAM-1 and ICAM-1. Chemokines produced at the sites of inflammation or injury act on endothelial cells to such that proteoglycans (such as heparan sulfate glycosaminoglycans) are expressed at high concentrations on their surface, whereas they activate leukocytes to convert low-affinity integrins such as VLA4 and LFA-1 to high-affinity state. These events ultimately lead to firm binding of activated leukocytes to activated endothelial cells such that leukocytes stop rolling, their cytoskeleton is reorganized, and they spread out on the endothelial surface. Binding of activated leukocytes to endothelial surface induces endothelial dysfunction and damage due to ROS, iNO produced by leukocytes. These adherent leukocytes migrate through interendothelial spaces towards the site of injury or infection by binding to certain molecules of the immunoglobulin superfamily called PECAM-1 (platelet endothelial cell adhesion molecule) or CD31. Leukocytes pierce the basement membrane by secreting collagenases, enzymes that can digest collagen.

One of the mechanisms by which leukocytes emigrate towards the sites of injury or inflammation is by a process called as chemotaxis that is induced by chemotaxins. These chemoattractants can be either endogenous or exogenous molecules. The most common exogenous chemoattractants are bacterial products, some of which are peptides that contain N-formyl-methionine terminal amino acid. Some of the endogenous chemoattractants include (but not limited to): components of the complement system such as C5a, lipoxygenase pathway products such as leukotriene B₄ (LTB₄), and some cytokines such as IL-8. Although the exact mechanism by which leukocytes sense and are attracted towards the chemosensory agents is not clear, studies suggested that majority of these chemoattractants bind to specific seven transmembrane G-protein-coupled receptors (GPCRs) on the surface of leukocytes [11]. GPCRs, in turn, activate phospholipase C (PLC), phosphoinositol-3-kinase (PI3K) and protein kinases. Both PLC and PI3K act on cell membrane phospholipids to generate lipid second messengers such as inositol triphosphate (IP3) that increase cytosolic calcium (Ca²⁺) and activate small GTPases of the Rac/Rho/cdc2 family as well as numerous kinases. GTPases induce polymerization of actin that helps in the motility of the leukocytes. In this context, it is interesting to note that Bucci et al [12] demonstrated that eNO synthase activation is critical for vascular leakage during acute inflammation. It was observed that in congenic eNO synthase-deficient (eNOS−/−) mice the early phase (0-6 hours) inflammation induced by intraplantar injection of carrageenan is eliminated, and the secondary phase (24-96 hours) of the inflammatory response is markedly reduced compared to WT (wild type) mice. Zymosan-induced inflammatory cell extravasation was similar in WT and eNOS−/− mice, whereas extravasation of plasma protein was lower in eNOS−/−mice. Inhibition of phosphatidylinositol 3-kinase and hsp90 also blocked protein leakage but not leukocyte influx [12]. These results clearly established the critical role of eNOS in vascular leakage during acute inflammation. But, it is not yet clear as to the exact relationship between selectins, VCAM-1 and ICAM-1, GPCRs, small GTPases of the Rac/Rho/cdc2 family as well as numerous kinases, and eNOS and how the interaction between these molecules influences the inflammatory process.

ii. Leukocyte activation: In order to kill microbes that produce inflammation, leukocytes generate ROS by a process that is termed as activation. Products of necrotic cells, antigen-antibody complexes, cytokines, and chemokines also induce leukocyte activation. Different classes of leukocyte cell surface receptors recognize different stimuli. For instance, chemokines, lipid mediators, and N-formyl-methionyl peptides increase integrin avidity, and produce cytoskeletal changes that aids leukocyte chemotaxis; microbial lipopolysaccharide (LPS) binds to toll-like receptors (TLRs) on leukocyte membrane leading to their activation and production of cytokines and ROS that are essential for the killing of microbes; and binding of microbial products to mannose receptor augments leukocyte phagocytic process that aids in the elimination of the invading organisms. Activation of leukocytes by various stimuli triggers several signaling pathways that result in increases in cytosolic Ca²⁺ and activation of protein kinase C (PKC) and phospholipase A₂ (PLA₂) that are ultimately seen in the form of various functional responses of leukocytes. In this context, it is interesting to note that PLA₂ activation leads to the release of lipids such as arachidonic acid (AA, 20:4Ω-6), eicosapentaenoic acid (EPA, 20:5Ω), and docosahexaenoic acid (DHA, 22:6Ω-3) from the cell membrane lipid pools. Studies showed that AA, and possibly EPA and DHA themselves could increase cytosolic Ca²⁺ and PKC concentrations in various cells [13, 14]. Furthermore, AA by itself has the ability to activate leukocytes [15]. These results suggest that simple dietary lipids have the ability to modulate leukocyte responses and the inflammatory process. Products of AA, EPA, and DHA such as prostaglandins (PGs), leukotrienes (LTs), lipoxins (LXs), and resolvins are also known to have both positive and negative influences on leukocyte activation, chemotaxis, inflammation and its resolution [16, 17]. Some of the products that are released by activated leukocytes include: AA and its metabolites, lysosomal enzymes, ROS, NO, various cytokines, various leukocyte adhesion molecules and other surface receptors such as TLRs, GPCRs, receptors for opsonins, etc.

iii. Phagocytosis and killing of microbes by ROS: In order to eliminate the invading microorganisms, leukocytes first have to phagocyte them and then release appropriate amounts of ROS and NO to kill them. Leukocytes use mannose receptors and scavenger receptors to bind and ingest bacteria, though they can engulf bacteria and other particles without attachment to specific receptors. Opsonins greatly enhance the efficiency of phagocytosis. Once the bacteria or other foreign particles are recognized by leukocytes, they are engulfed for killing them.

Killing and degradation of the ingested bacteria or foreign particles both by leukocytes and macrophages is accomplished by ROS, NO, and ozone. In general, phagocytosis stimulates NADPH oxidase accompanied by a burst of oxygen consumption, glycogenolysis, and increased glucose oxidation via the hexose-monophosphate shunt pathway. ROS, NO and ozone have the ability to kill bacteria. The azurophilic granules of neutrophils contain myeloperoxidase (MPO), which, in the presence of a halide such as Cl⁻, converts H₂O₂ to hypochlorite (HOCL). HOCL is a potent antimicrobial agent by binding covalently to cellular constituents or by oxidation of proteins and lipids [18]. Once leukocytes have performed their function of killing the bacteria, they rapidly undergo apoptosis and are ingested by macrophages.

It should be noted that bacterial killing could also occur by oxygen-independent mechanisms. For instance, hitherto it is believed that neutrophils kill ingested microorganisms by releasing high concentrations of ROS and bringing about myeloperoxidase-catalyzed halogenation as described above. In a recent study, Reeves et al [19] showed that mice made deficient in neutrophil-granule proteases but normal in respect of ROS production and iodinating capacity are unable to resist staphylococcal and candidal infections. They showed that activation of neutrophils provokes the influx of high amounts of ROS into the endocytic vacuole that results in an accumulation of anionic charge that is compensated by a surge of K⁺ ions. These K⁺ ions cross the membrane in a pH-dependent manner inducing a steep rise in ionic strength that results in the release of cationic granule proteins, including elastase and cathepsin G. It is the release of these proteases that is primarily responsible for the destruction of bacteria. Thus, there appears to be a close relationship between ROS and the release of proteases, and bactericidal action of neutrophils. But, it looks as though; proteases are primarily responsible for bactericidal action but not ROS themselves. These observations have important clinical implications since, the relative importance of MPO and NADPH oxidase generated ROS in fight against various infections is a contentious issue. Aratani et al [20] demonstrated that mice that have no MPO activity in their neutrophils and monocytes developed normally, were fertile, and showed normal clearance of Staphylococcus aureus. However, these animals showed increased susceptibility to Candida albicans infection. Furthermore, lack of MPO significantly enhanced the dissemination of Candida albicans into various organs. These results suggest that MPO is important for early host defense against fungal infections. In contrast, the same authors reported that both MPO (MPO−/−) and NADPH oxidase deficient (X-linked chronic granulomatous disease, X-CGD) mice are susceptible to pulmonary infections with Candida albicans and Aspergillus fumigatus compared with normal mice, and the X-CGD mice exhibited shorter survival than MPO−/− mice [21]. This increased mortality in the X-CGD mice was associated with a 10- to 100-fold increased outgrowth of the fungi in their organs. These results suggest that O₂.⁻ produced by NADPH oxidase is more important than HOCL produced by MPO against pulmonary infection with those fungi. It is interesting to note that at the highest dose of Candida albicans, the mortality of MPO−/− mice was comparable to X-CGD mice, but was the same as normal mice at the lowest dose [22]. At the middle dose, the number of fungi disseminated into various organs of the MPO−/− mice was comparable to the X-CGD mice in one week after infection, but it was significantly lower in 2 weeks. These results suggest that both MPO and NADPH oxidase are equally important for early host defense against large inocula of Candida albicans. Hereditary MPO deficiency is common that has an estimated incidence of 1 in 2,000 in the United States. The results of the studies performed by Aratani et al [20-22] suggest that MPO-deficient individuals could exhibit similar problems as CGD patients if exposed to a large amount of fungi/microorganisms. It is likely that MPO deficient diabetics are more susceptible to fungal infections, if the dose of inocula is small, compared to normal.

3. Mediators of Inflammation

There are many chemical mediators of inflammation. Although the exact function and the source of some of the chemical mediators are not very clear, certain generalizations are possible. It should also be noted that there could be some as yet unidentified chemical mediators of inflammation. Some of the important mediators of inflammation include: histamine, serotonin, lysosomal enzymes, PGs, LTs, PAFs, ROS, NO, HOCL, various cytokines, kinin system, coagulation/fibrinolysis system, and complement system. Some of the general properties of the mediators of inflammation are given below.

Plasma-derived mediators such as complement proteins and kinins are present in plasma in precursor forms that must be activated by a series of proteolytic cleavages, to acquire their biologic properties. On the other hand, cell-derived mediators need to be secreted (e.g., histamine in mast cell granules) or are synthesized de novo (e.g., prostaglandins, cytokines) in response to a given stimulus. The major cellular sources of these mediators are platelets, neutrophils, monocytes/macrophages, and mast cells, but mesenchymal cells such as endothelium, smooth muscle, fibroblasts, and most epithelia can also be induced to elaborate some of these mediators. The invading microorganisms trigger the production of most of these mediators or host derived products such as complement, kinins, etc., that are themselves activated by microbes or tissues under attack. These mediators, generally, bind to their specific receptors on target cells to produce their actions. In some instances, they themselves have direct enzymatic activity or induce the production of reactive oxygen species (ROS) or nitric oxide (NO) that, in turn, either mediate their actions or induce tissue damage. It is also interesting to note that in majority of the instances, one mediator triggers the release of another mediator that acts on the target tissue. These secondary mediators either potentiate the action of the initial mediator or paradoxically abrogate its action. Thus, the ultimate degree of inflammation depends on the balance between such pro- and anti-inflammatory mediators. In some instances, the anti-inflammatory chemicals or signals initiated may not only act on the target tissue but also on other tissues to suppress inflammation. Thus, both pro- and anti-inflammatory mediators may act on specific tissues or diverse and tissues. Once released or activated, most of these mediators are inactivated or decay quickly. For instance, arachidonic acid and its metabolites have a short half-life, whereas specific or non-specific enzymes inactivate kinins. On the other hand, ROS and NO are scavenged by specific or non-specific antioxidants [9]. This suggests that under normal physiological conditions, there are both positive and negative checks and balances and when an imbalance sets in this well-balanced system pathological events occur.

Histamine, serotonin, bradykinin, complement system and coagulation cascade are too well known to be discussed here and their involvement in infections, inflammatory process and sepsis and septic shock is well known.

b. Platelet Activating Factor (PAF)

PAF is another bioactive phospholipid-derived mediator. It is known to have multiple pro-inflammatory effects. Chemically, PAF is acetyl-glyceryl-ether-phosphorylcholine (AGEPC), a phospholipid with a glycerol backbone, a long-chain fatty acid in the A position, an unusually short chain substituent in the B location, and a phosphatidylcholine moiety. PAF mediates its effects via a single G-protein-coupled receptor, and a family of inactivating PAF acetylhydrolases regulates its effects. Platelets, basophils, mast cells, neutrophils, monocytes/macrophages, and endothelial cells can elaborate PAF. PAF not only causes platelet activation but also causes vasoconstriction and bronchoconstriction, and at extremely low concentrations induces vasodilatation and increased venular permeability with potency many times greater than that of histamine. PAF also causes leukocyte adhesion to endothelium by enhancing integrin-mediated leukocyte binding, chemotaxis, degranulation, and the oxidative burst. PAF boosts the synthesis of eicosanoids by leukocytes and other cells. Thus, PAF can elicit all the cardinal features of inflammation [23]. PAF receptor antagonists inhibit inflammation in some experimental models.

c. Cytokines and Chemokines in Inflammation

Cytokines are proteins produced by many cell types including activated lymphocytes and macrophages, endothelial cells, epithelial cells, and connective tissue cells and have the ability to modulate the functions of various other cells. Cytokines not only have a regulatory role in cellular immune responses but also participate in both acute and chronic inflammation. TNF, IL-1, and IL-6 are the major cytokines that are involved in inflammation and have pro-inflammatory actions. On the other hand, IL-4 and IL-10 have anti-inflammatory actions, restrict inflammation and thus, they antagonize the actions of IL-1, IL-6 and TNF-α. Activated macrophages and T cells produce them. But recent studies showed that a variety of other cells and tissues are also capable of producing these cytokines. For instance, endothelial cells, adipose tissue, Kupffer cells, and glial cells are capable of producing them. Endotoxin and other microbial products, immune complexes, physical injury, and a variety of inflammatory stimuli can stimulate the secretion of TNF and IL-1. They activate endothelial cells, stimulate leukocytes, and fibroblasts, and induce systemic acute-phase reactions. Activation of endothelial cells by TNF, IL-6, and IL-1 induces a spectrum of changes-mostly regulated at the level of gene transcription, and induce the synthesis of endothelial adhesion molecules and chemical mediators of inflammation such as other cytokines, chemokines, growth factors, eicosanoids, and nitric oxide (NO) [24]. These events increase the thrombotic tendency on the surface of the endothelium. TNF primes neutrophils, leading to augmented responses of these cells to other mediators, and stimulates neutrophils to produce ROS [25]. IL-1, IL-6, and TNF-α induce the systemic acute-phase responses associated with infection or injury such as fever, loss of appetite, slow-wave sleep, the release of neutrophils into the circulation, the release of corticotropin and corticosteroids. When large amounts of these cytokines are released they may produce hemodynamic effects of septic shock such as hypotension, decreased vascular resistance, increased heart rate, and decreased blood pH that may ultimately cause death. Sustained and increased production of TNF-α as it occurs during chronic intracellular infections such as tuberculosis and neoplastic diseases lipid and protein mobilization occurs leading to the development of cachexia in these patients. IL-1, IL-6, and TNF-α suppress appetite and this contributes to cachexia [26]. Increased production of IL-1, IL-6, and TNF-α is also seen in rheumatoid arthritis and systemic lupus erythematosus (SLE), and other collagen vascular diseases. This discovery led to the development anti-TNF-α antibodies and TNF-α receptor blockers that found their use in the treatment of these conditions.

d. Low-Grade Systemic Inflammation in Metabolic Syndrome X

Recent studies suggested that low-grade systemic inflammation plays a significant role in the pathogenesis of type 2 diabetes [27, 28]. This is based on the observation that the plasma concentrations of C-reactive protein (CRP), TNF-α, IL-6, and resistin, which are markers of inflammation, are elevated whereas the concentrations of adiponectin that shows anti-inflammatory actions are reduced in type 2 diabetes mellitus [29-31].

Several other studies also revealed that elevated plasma concentrations of CRP and possibly, IL-6 and TNF-α predict the future development of type 2 diabetes mellitus, hypertension, and coronary heart disease [32-34]. Furthermore, reduction in the levels of CRP, IL-6 and TNF-α achieved by diet control, exercises, and statin therapy predicted a better outcome to these patients. This suggests that measurement of these inflammatory markers could be used to predict the development of metabolic syndrome and response to various therapies.

f. Chemokines

Chemokines are a family of small (8 to 10 kD) proteins that act primarily as chemoattractants for specific types of leukocytes [35-37]. In all, about 40 different chemokines and 20 different receptors for chemokines have been identified. They are classified into four major groups, according to the arrangement of the conserved cysteine (C) residues in the mature proteins. Chemokines mediate their action by binding to seven transmembrane G-protein-coupled receptors that usually exhibit overlapping ligand specificities, and leukocytes generally express more than one receptor type. Certain chemokine receptors (eg. CXCR-4, CCR-5) act as co-receptors for a viral envelope glycoprotein of human immunodeficiency virus (HIV-1) and are thus involved in binding and entry of the virus into cells. Chemokines have the ability to stimulate leukocyte recruitment in inflammation and control the normal migration of cells through various tissues [38]. Some chemokines are produced transiently in response to inflammatory stimuli and promote the recruitment of leukocytes to the sites of inflammation, whereas others are produced constitutively in tissues and participate in organogenesis. In both situations, chemokines are displayed at high concentrations attached to proteoglycans on the surface of endothelial cells and in the extracellular matrix.

g. Nitric Oxide (NO)

NO was originally discovered as a factor that is released from endothelial cells that caused vasodilatation and hence was called as endothelium-derived relaxing factor [39]. NO is a soluble gas that is produced not only by endothelial cells, but a variety of cells such as macrophages and neurons in the brain. It is now evident that many cells (if not all) produce NO and that it also participates in inflammation. NO acts in a paracrine manner on target cells through induction of cyclic guanosine monophosphate (cGMP) that, in turn, initiates a series of intracellular events leading to the desired response such as relaxation of vascular smooth muscle cells, neurotransmission, tumoricidal, cytotoxic, and bactericidal actions. The half-life of NO is only few seconds and hence, it has to be produced in close proximity to where it is needed.

NO is synthesized from L-arginine by the action of nitric oxide synthase (NOS) enzyme [40]. There are three different types of NOS-endothelial (eNOS), neuronal (nNOS), and inducible (iNOS). NOS exhibit two patterns of expression: eNOS and nNOS are constitutively expressed at low levels and can be activated rapidly by an increase in cytoplasmic calcium ions. Influx of calcium into cells leads to a rapid production of NO. In contrast, iNOS is induced in macrophages and other cells when are activated by cytokines such as TNF-α and IFN-γ. It is paradoxical to know that eNO and nNO have many beneficial properties whereas iNO shows pro-inflammatory actions.

NO plays an important role in the vascular and cellular components of inflammatory responses. NO is a potent vasodilator and prevents platelet aggregation. NO inhibits vascular smooth muscle cell proliferation. NO reduces platelet adhesion and inhibits several features of mast cell-induced inflammation, and serves as an endogenous regulator of leukocyte recruitment. Inhibition of endogenous NO production promotes leukocyte rolling and adhesion in postcapillary venules. On the other hand, delivery of exogenous NO reduces leukocyte recruitment. Thus, under normal physiological conditions NO is an inhibitor of inflammatory response and possibly, increased production of NO in inflammatory conditions could be a compensatory mechanism to block inflammatory responses [41]. But, it should be understood that increased production of NO seen in response to various inflammatory stimuli might itself perpetuate inflammation. This is so since in these situations NO may get converted to peroxynitrite radical that has potent pro-inflammatory actions. Decreased production of eNO occurs in insulin resistance, obesity, atherosclerosis, diabetes, and hypertension [42-45].

NO and its derivatives have microbicidal actions and thus, NO functions as an endogenous mediator of host defense against infections [46]. This is supported by the observation that: (a) reactive nitrogen intermediates derived from NO possess antimicrobial activity; (b) NO interacts with ROS to form multiple antimicrobial metabolites; (c) in response to infections the production of NO is increased by macrophages and other immune cells; and (d) inactivation of iNOS enhances the incidence of infections and augments the multiplication of microbial organisms in experimental animals. Enhanced production of NO by macrophages and other immune cells has been shown to inhibit the growth of several bacteria, viruses, fungi, and other organisms. It is relevant to note that NO also had tumoricidal actions.

Although NO is unstable, its concentrations in the plasma and various cells in vitro could be measured using various colorimetric techniques and specific NO probes. NO is measured as its stable metabolites nitrite and nitrate in the plasma that gives an indication as to the concentrations of NO that is released by endothelial cells. Highly sensitive NO probes are commercially available to measure intracellular concentrations of NO and NO that is released by cells in vitro cultures. These techniques allow one to study various factors that regulate NO production. These techniques enable one to study the effect of various chemicals, drugs, and factors that influence the generation of NO. Thus, it is now possible to assess the generation of NO in vivo and in vitro by various cells and tissues.

h. Leukocyte Lysosomal Enzymes

Lysosomal granules present in neutrophils and monocytes are of two types: smaller specific (secondary) granules and larger azurophil (primary) granules. The smaller specific secondary granules contain lysozyme, collagenase, gelatinase, lactoferrin, plasminogen activator, histaminase, and alkaline phosphatase. On the other hand, the large azurophil primary granules contain myeloperoxidase, lysozyme, defensins, acid hydrolases, and a variety of neutral proteases such as elastase, cathepsin G, proteinase 3, and nonspecific collagenases [47]. Both types of granules release their contents into phagocytic vacuoles that form around engulfed material to bring about their actions. These granule contents can also be released into the extracellular space. The release of the contents of lysosomal granules contributes to inflammation. It may be noted here that different granule enzymes show different functions. For instance, acid proteases degrade bacteria and debris within the phagolysosomes under acidic pH conditions, whereas neutral proteases degrade various extracellular components. Neutral proteases attack and degrade collagen, basement membrane, fibrin, elastin, and cartilage that ultimately result in tissue destruction that are typically seen in acute and chronic inflammatory processes. Neutral proteases have the ability to cleave C3 and C5 directly resulting in the release of anaphylatoxins, and kinin-like peptide from kininogen. Neutrophil elastase degrades virulence factors of bacteria and thus helps in the control of bacterial infections [48]. Both monocytes and macrophages contain acid hydrolases, collagenase, elastase, phospholipase, and plasminogen activator by virtue of which they participate in chronic inflammatory reactions. In view of the destructive nature of lysosomal enzymes that are released by neutrophils, it is important that methods should be designed to control leukocytes infiltration at the site of injury and infection. If the leukocyte infiltration remains unchecked, it can lead to further increase in vascular permeability and tissue destruction. In order to control the harmful effects of these proteases, a number of antiproteases are present in the serum and tissue fluids. One of the best examples of such an antiproteases is α₁-antitrypsin that inhibits neutrophil elastase. A deficiency of α₁-antitrypsin leads to uncontrolled action of leukocyte elastase that is known to be associated with pulmonary damage resulting in emphysema. α₂-macro-globulin is another antiprotease found in serum and various secretions.

i. Reactive Oxygen Species (ROS)

ROS or oxygen-derived free radicals are released by leukocytes, macrophages and other similar cells present in various organs into the extracellular compartment on exposure to various noxious agents such as microbes, foreign objects, and in response to chemokines, ingestion of immune complexes, or following a phagocytic challenge [49]. The production of ROS is due to the activation of the NADPH oxidative system. Known ROS species are mainly: superoxide anion (O₂⁻.), hydrogen peroxide (H₂O₂), and hydroxyl radical (OH). ROS are produced mainly within the cell, and are capable of reacting with NO to form reactive nitrogen intermediates that are cytotoxic to various organelles of cells [50]. Since ROS and reactive nitrogen intermediates are highly toxic, their release into the extracellular space even in low concentrations may prove to be harmful. Furthermore, even at very low concentrations they are capable of increasing the expression of chemokines (e.g., IL-8), cytokines, and endothelial leukocyte adhesion molecules, events that are capable of amplifying the inflammatory cascade [51]. The physiological function of both ROS and reactive nitrogen species are capable of destroying bacteria, viruses, fungi, and cancer cells. At the other end of the spectrum, increased production of ROS and reactive nitrogen intermediates are potentially harmful and could cause acute and chronic inflammation, sepsis, and other pathological conditions. Thus, ROS and reactive nitrogen intermediates (RNI) can cause endothelial cell damage that results in increased vascular permeability, insulin resistance, and thrombosis. In this context, it is important to note that activated adherent neutrophils not only produce ROS and RNI but also stimulate xanthine oxidase in endothelial cells that, in turn elaborates further generation of superoxide anion. ROS and RNI inactivate antiproteases such as α₁-antitrypsin that leads to unopposed protease activity, which could result in increased destruction of extracellular matrix. ROS by themselves damage many cells and tissues including but not limited to parenchymal cells. It is now believed that several clinical conditions are due to excess production of ROS. For instance, there is reasonable evidence to suggest that ROS and RNI are responsible for diseases such as rheumatoid arthritis, lupus, and other collagen vascular diseases; ulcerative colitis, ischemia-reperfusion injury to myocardium following coronary bypass surgery and cerebral cortical damage after ischemic stroke; and several pathophysiological processes such as insulin resistance, metabolic syndrome X, atherosclerosis, schizophrenia, Alzheimer's disease, etc. In view of this, efforts are being made to develop anti-oxidants and free radical quenchers that might mitigate these diseases and processes. There is also evidence available to indicate that various features of metabolic syndrome X are due to low-grade systemic inflammation that in turn is due excess production of ROS and RNI in specific tissues in question. For instance, excess production of ROS in endothelial cells (or close to endothelial cells) produce damage to these cells that results in endothelial dysfunction. Obesity, hypertension, type 2 diabetes mellitus, hyperlipidemias, and CHD, which are components of metabolic syndrome X, are all characterized by endothelial dysfunction. This view is supported by the fact that increased generation of ROS is seen in obesity, hypertension, type 2 diabetes mellitus, hyperlipidemias, and insulin resistance. But, it is not yet clear why and how increased generation of ROS occurs. Once the exact reason or the stimulus that is responsible for ROS in these conditions is identified, it will be possible to develop reasonable therapeutic approaches to prevent or even treat these conditions. It is important to know as to when this increase in the generation of ROS starts so that appropriate timing of preventive or therapeutic measures is known.

In order to abrogate the harmful actions of ROS, several antioxidants are present in the serum, various tissue fluids, and cells. These antioxidants include: (1) the copper-containing serum protein ceruloplasmin; (2) the iron-free fraction of serum, transferrin; (3) the enzyme superoxide dismutase (SOD), which is found or can be activated in a variety of cell types; (4) the enzyme catalase, which detoxifies H₂O₂; and (5) glutathione peroxidase, another powerful H₂O₂ detoxifier. Thus, the influence of ROS in inflammatory conditions depends on the balance between the production and the inactivation of these metabolites by cells and tissues.

With the identification of NO, it is clear that it also has an important role in the pathogenesis of both acute and chronic inflammation. Excess production of NO, especially by macrophages is harmful to several tissues. Activation of iNOS that occurs in response to various stimuli by itself sometimes is sufficient to initiate and perpetuate the inflammatory process. But, more often than not, excess production of both ROS and NO occurs in majority of the inflammatory conditions and yet times it is extremely difficult to separate individual role of ROS and NO in a given pathology or inflammatory condition.

It is important to note that NO has many useful actions as well. NO is a potent platelet anti-aggregator and vasodilator and has been thought to prevent atherosclerosis. Production of appropriate amounts of eNO is possible only when endothelial cells are healthy. Hence, plasma concentrations or endothelial production of NO can be used as a marker of endothelial cell integrity and health. In obesity, hypertension, type 2 diabetes mellitus, insulin resistance, hyperlipidemias, and CHD, the plasma concentrations of NO are low that suggests that endothelial dysfunction is present in all these conditions. NO levels revert to normal following weight loss achieved by diet restriction and exercise, control of hypertension, normalization of plasma glucose levels in type 2 DM, and reduction of plasma lipid levels. Thus, measurement of plasma levels of NO could be used as a marker not only of endothelial function but also to judge adequacy of treatment given to patients in these conditions. Since many factors could influence the synthesis and half-life of NO, it is important to keep a note of them. For instance, decreased production of NO could be due to a deficiency of its precursor, L-arginine, and/or lack or deficiency of co-factors such as tetrahydrobiopterin (BH₄) [52]. Hence, at times simple lack or deficiency of these co-factors may lead to low plasma levels of NO. Hence, before a judgment as to the cause of decreased NO levels is made, one has to take these factors into consideration.

j. Neuropeptides in Inflammation

Neuropeptides are known to play a significant role in the initiation and propagation of inflammation. Substance P and neurokinin A that are produced both in the central and peripheral nervous systems have the ability to influence transmission of pain signals, regulation of blood pressure, stimulation of secretion by endocrine cells, and increasing vascular permeability [53-55]. The involvement of these neuropeptides in the inflammatory process explains the neurogenic component of inflammation. Sensory neurons produce certain pro-inflammatory molecules that link the sensing of dangerous stimuli to the development of protective host responses that form the basis of neurogenic inflammation [55].

4. Present Clinical Laboratory Tools to Diagnose Inflammation

It is evident from the preceding discussion that many biological molecules are involved in the pathobiology of inflammation. At bedside, it is relatively simple to diagnose acute inflammation that is characterized by rubor, tumor, calor, dolor, and functiolaesa (redness, swelling, heat, pain, and loss of function respectively). Since these acute inflammatory events are easily visible, perhaps, no specific laboratory tests are necessary to measure the presence or absence of inflammation. But, when the inflammatory process is low-grade and localized to the internal organs it is difficult, if not impossible, to detect and confirm the presence of inflammation. This is especially true when there is low-grade systemic inflammation. When chronic inflammation occurs as a result of infections or infestations, it calls for specific tests. For example, in subjects who have chronic malaria (especially when it is due to Plasmodium malariae and ovale) and when it occurs in partially immune individuals, it is extremely difficult to diagnose the disease. This is so, since the classical signs of malaria such as fever with chills and rigors do not manifest themselves clearly. In such an instance, one has to carefully screen the peripheral blood smear for the malarial organism. But even the screening of peripheral blood smear is ordered only when the clinician suspects the presence of malaria. In view of this, one has to have a high degree of clinical suspicion even to order for the peripheral smear examination for malarial organisms. On clinical grounds, the clinician will be able to suspect the presence of malarial infection in an individual only when one finds significant hepatosplenomegaly, loss of weight and appetite, and whether the patient is hailing from an endemic area of malaria or has been recently to a tropical country where malaria is common. On the other hand, in this modern era wherein the incidence of infections is becoming less common whereas degenerative conditions and geriatric diseases are more frequent it is becoming increasingly difficult to diagnose diseases in which low-grade systemic inflammation is common. Examples of diseases in which low-grade systemic inflammation is common include: obesity, insulin resistance, type 2 diabetes mellitus, hypertension, coronary heart disease (CHD), dyslipidemia, atherosclerosis, various cancers, and dormant chronic inflammatory conditions such as rheumatoid arthritis (RA), systemic lupus erythematosus (SLE), progressive systemic sclerosis (PSS), mixed connective tissue disorder (MCTD), vasculitis; and other disorders caused by uncontrolled angiogenic activity such as proliferative diabetic retinopathy; other eye disorders such as macular degeneration; and central nervous system disorders such as multiple sclerosis, Alzheimer's disease; skin problems such as psoriasis, renal conditions such as chronic renal failure, end-stage renal disease, glomerulonephritis such as minimal change nephropathy, various forms of proliferative glomerulonephritis, nephritis secondary to underlying systemic diseases such as collagen vascular diseases, lymphoma and leukemias. The belief that inflammation plays a significant role in these conditions has come from the observation that subjects with these diseases have enhanced plasma levels of CRP, IL-6, and TNF-α. These patients also show low circulating NO levels and simultaneously increased generation of reactive oxygen species (ROS). Increased ROS decreases anti-oxidant content of the cells/tissues due to their utilization. Thus, these patients may show decreased vitamin E, superoxide dismutase, and glutathione levels. This suggests that ultimately the delicate balance between the pro- and anti-oxidant status is tilted more in favor of the pro-oxidants leading tissue damage and disease. But, it is not yet certain as to what actually triggers the initiation of the disease process. Once this is understood, perhaps, it is possible to take adequate steps or device methods/drugs to stop the development of the disease process.

Recently, a number of studies showed that other inflammatory markers could be used to predict the development of various cardiovascular diseases, atherosclerosis, and other diseases enumerated above and to predict their prognosis. Interleukin-1 (IL-1), IL-6, IL-8, IL-10, tumor necrosis factor-α (TNF-α), high-sensitive CRP, and monocyte chemoattractant protein (MCP-1) are some such factors that have been studied. Both adhesion molecules such as intracellular adhesion molecule-1 (ICAM-1) and soluble vascular adhesion molecule-1 and proinflammatory cytokines IL-1, IL-6, IL-8, IL-10, and TNF-α have been associated with a risk of new coronary events in ischemic heart diseases and with clinical recurrence of symptoms [56-61]. But, these markers are relatively less dependent. This so because, these markers are relatively unstable in serum, serum and plasma samples need to be rapidly separated from the cellular constituents of blood, and assayed rapidly or the samples need to be frozen to prevent degradation of the cytokines and adhesion molecules. Typically, these assays are performed using ELISA technique. If more automated assay methods become available, then perhaps their assay may become more popular. Multiplex assays for several cytokines are also being developed that shows great promise.

Another issue with most of the cytokine assays is that sometimes they show imprecision. The cytokine assays also lack a sufficiently low limit of quantification for use in apparently healthy subjects. But as advances occur, more accurate and precise methods to quantify cytokine levels within the reference interval (i.e., the concentrations encountered in an apparently healthy population) may become available so that they could be measured more reliably.

Recent studies showed that fibrinogen was consistently associated with long-term risk [62], although its association differs among studies. This in part could be due to the differences in the analytical methods employed. Recently, serum amyloid A was observed to be a more reliable marker in CHD [63, 64], although some of these results have been inconsistent. In one study, serum amyloid A but not hs-CRP was found to be associated with the extension of CHD, suggesting that both markers have a similar association with events but may possess different roles in the pathogenesis of atherosclerosis but not in the prediction of future events.

IL-18, originally described as interferon-inducing factor, is present atherosclerotic plaques [65]. IL-18 has been shown to be associated with future cardiovascular death in a 3.9-year-long follow-up of patients with stable angina and unstable angina pectoris. The predictive value of IL-18 was similar to that of hs-CRP, suggesting that it does not add significant value in predicting future CHD compared to hs-CRP [66].

Myeloperoxidase is a pro-inflammatory leukocyte enzyme that is present in abundant amounts in the ruptured plaque. Recent studies showed that myeloperoxidase could be associated with the recurrence of CHD and other cardiovascular events even in those who were negative for troponins [67]. It is also interesting to note that the predictive value of myeloperoxidase was found to be independent of both troponin and hs-CRP levels [68]. It remains to be seen whether myeloperoxidase could be used routinely to predict prognosis of patients with CHD.

Other But More Conventional Markers of Inflammation

Leukocytosis is known to be an excellent marker of inflammation. Recent studies revealed that higher leukocyte count could be associated with a greater cardiovascular risk. Since there are many extraneous factors that can influence leukocyte count, one need to be careful in using leukocyte count as a marker of predicting or prognosticating cardiovascular risk. For instance, leukocyte count has to be done on fresh specimens, current cigarette smoking increases leukocyte count; any unnoticed or sub-clinical infections also increase leukocyte counts limiting its utility.

Elevated fibrinogen levels have been shown to be a major independent risk factor for cardiovascular diseases and stroke outcomes [69, 70]. Higher fibrinogen levels enhanced the CHD risk of patients with hypertension, cigarette smokers, and people with diabetes.

Necessity of More Reliable and Dependable New Markers of Inflammation

It is evident from the preceding discussion that inflammation is a complex process or phenomena and it is extremely difficult to pinpoint the chemical(s) that start the whole process. With advances in molecular biology and laboratory techniques, it is apparent that our understanding of fundamentals of inflammation has improved. But these advances are not yet sufficient to help us to devise better methods of detecting and treating inflammation, especially low-grade systemic inflammation that is seen in CHD, metabolic syndrome X, hypertension, etc. Nevertheless, recent studies showed that low-grade inflammation plays a significant role in many, hitherto believed to be degenerative conditions. Since inflammation is a fundamental process of all living organisms, it remains to be seen how it can influence several other cellular processes such as longevity, cancer, etc. There is significant amount of data available to suggest that inflammation has a role in the pathogenesis of cancer. But, it is not yet certain how and where it starts to initiate the cancerous process. In this context, it is interesting to note that platelets also play a significant role in inflammation especially markers of platelet activation such as RANTES and in particular CD40L The fact that platelets, in some unknown way, participate in metastasis of tumor cells once again underscores the relationship between inflammation and various diseases processes. Platelet anti-aggregators have been shown to either prevent or substantially reduce tumor cell metastasis in experimental animals thought this did not find an application in clinical practice. This once again reminds the grim fact that there is a big gap between results obtained in animal studies and the clinic. But, it is believed with fond hope that advances made in the lab would eventually find their application in the clinic. This is evident from the fact that a better understanding of inflammation would eventually lead to the use of either existing or newer anti-inflammatory compounds could be of significant benefit in several conditions such as cardiovascular diseases, cancer, and stroke.

The observation that acute, chronic and low-grade systemic inflammation plays a role in various diseases underscores the importance of developing newer laboratory tests for their detection.

In this context, we observed that butyrylcholinesterase could be a new and reliable method of detecting and diagnosing the existence of low-grade systemic inflammation, and acute and chronic inflammatory events in all the above-mentioned conditions using plasma and/or tissue levels of butyrylcholinesterase as a marker.

Cholinesterase and Butyrylcholinesterase

There are at least two choline esterases. Acetylcholinesterase is a specific choline esterase, hydrolyzing predominantly choline esters, and characterized by high concentrations in brain, nerve and red blood cells (RBCs). The other type, called butyrylcholinesterase, is a nonspecific-choline esterase (also called as “pseudo” choline esterase) hydrolyzing other esters as well as choline esters, and found in blood serum, pancreas, liver, and central nervous system [71, 72]. Specific choline esterase develops its maximum activity at pH 7 and at low levels of acetylcholine (less than 2.5 mg %). Both enzymes are inhibited by very small quantities of physostigmine. Phosphorous-containing insecticides and nerve gases inhibit acetylcholinesterase.

Activity of acetylcholine in the brain is terminated by the hydrolytic action of cholinesterases. Inhibitors of these enzymes (cholinesterases) have hence been developed to augment the activity of cholinergic neurons in the brain. Such an effort is useful especially in patients with Alzheimer's disease, who have decreased forebrain cholinergic neurons and a progressive decline in acetylcholine (73, 74). All cholinesterase inhibitors currently licensed for Alzheimer's disease inhibit, Acetylcholinesterase (ACHE, EC 3.1.1.7) and to a varying degree, butyrylcholinesterase (BChE, EC 3.1.1.8), which is a second cholinesterase in the brain [75]. Acetylcholinesterase and butyrylcholinesterase have numerous physiological functions depending on their localization and time of expression [76].

The classical action of Acetylcholinesterase is to catalyze hydrolysis of acetylcholine within cholinergic synapses of the brain and autonomic nervous system [77]. Although butyrylcholinesterase shares some of these functions, its role in brain remains unclear. To define its role in brain, selective inhibitors were designed and synthesized based on the X-ray crystallographic structure of the binding sites for acetylcholine that differentiate between butyrylcholinesterase and acetylcholinesterase [78].

In healthy human brain, Acetylcholinesterase predominates over butyryl-cholinesterase, but the latter likely has been previously underestimated [79]. Whereas, histochemically, acetylcholinesterase is localized mainly to neurons, butyryl-cholinesterase is associated primarily with glial cells, as well as to endothelial cells and neurons [80]. An important feature distinguishing butyrylcholinesterase from acetylcholinesterase is its kinetic response to concentrations of acetylcholine; reflected in their Kₘ values. Butyrylcholinesterase is less efficient in acetylcholine hydrolysis at low concentrations but highly efficient at high ones, at which acetylcholinesterase becomes substrate inhibited [81]. Hence, a possible role for brain butyrylcholinesterase, particularly when associated with glia, is for supportive hydrolysis of acetylcholine. Under conditions of high brain activity, local synaptic acetylcholine can reach micromolar levels that approach inhibitory levels for acetylcholinesterase activity. The close spatial relationship of glial butyrylcholinesterase would allow synergistic butyrylcholinesterase-mediated hydrolysis to assist in the regulation of local acetylcholine levels to permit the maintenance of normal cholinergic function. The survival of acetylcholinesterase knockout mice [79] with normal levels and localization of butyrylcholinesterase [82] supports the concept that butyrylcholinesterase has a key role that can partly compensate for the action of acetylcholinesterase.

Cholinesterase and Butyrylcholinesterase in Alzheimer's Disease

This alteration in the levels of butyrylcholinesterase in situations wherein there is a deficiency or absence of acetylcholinesterase assumes significance in clinical conditions in which acetylcholinesterase deficiency becomes significant such as Alzheimer's disease. For instance, in Alzheimer's disease, acetylcholinesterase is lost early up to 85% in specific brain regions, whereas butyrylcholinesterase levels, chiefly the G₁ form, rise with disease progression [73, 83]. The ratio of butyrylcholinesterase to acetylcholinesterase changes dramatically in cortical regions affected by Alzheimer' disease from 0.2 up to as much as 11 [84]. This altered ratio in Alzheimer's disease brain will modify the normally supportive role of butyrylcholinesterase in hydrolyzing excess acetylcholine only. Selective butyrylcholinesterase inhibition may therefore be useful in ameliorating a cholinergic deficit, which likely worsens in Alzheimer's disease due to increased activity of butyrylcholinesterase.

Histochemical studies revealed that some cholinergic neurons contain butyrylcholinesterase instead of acetylcholinesterase [85]. In fact, 10-15% of cholinesterase-positive cells in human amygdala and hippocampus are regulated by butyrylcholinesterase independently of acetylcholinesterase [86]. Augmenting cholinergic function by inhibiting these pathways (especially of butyrylcholinesterase in Alzheimer's disease) may be of clinical value. This is supported by the observation that rivastigmine, a dual cholinesterase inhibitor, improves cognitive function in patients with Alzheimer's disease, lends support to the concept that inhibition of butyrylcholinesterase in addition to acetylcholinesterase is of significant clinical benefit in Alzheimer's disease [87].

In the Alzheimer's disease brain, increasing levels of butyrylcholinesterase correlate significantly and positively with the development of hallmark cortical and neocortical amyloid-rich neuritic plaques and neurofibrillary tangles [74, 88]. Although the precise role of β-amyloid peptide, which accumulates in neuritic plaques, is not well understood, it is believed that it is toxic to neurons. Therefore, reducing β-amyloid peptide synthesis is a major focus of current Alzheimer's disease research.

Since there is a role for butyrylcholinesterase in central cholinergic transmission, its expression is altered in Alzheimer's disease brain, and its probable association with the development of neuropathologic changes seen in Alzheimer's disease, efforts is being made to inhibit the activity of butyrylcholinesterase since high butyryl-cholinesterase activity would be detrimental in Alzheimer's disease. In support of this contention, Greig et al [89] reported that in rat brain slices selective butyrylcholinesterase inhibition augmented long-term potentiation, improved the cognitive performance of aged rats, and in cultured human SK-N-SH neuroblastoma cells, intra-n and extracellular β-amyloid precursor protein, and the secreted β-amyloid peptide levels were found to be reduced. In addition, it was also observed that treatment of transgenic mice that overexpressed human mutant amyloid precursor protein also resulted in lower β-amyloid peptide brain levels than controls. These results indicate that selective inhibition of brain butyryl-cholinesterase could form a treatment for Alzheimer's disease by improving cognition and modulating neuropathological markers of the disease. These results also imply that estimation of acetylcholinesterase and butyrylcholinesterase could be used as possible markers of Alzheimer's disease and other diseases in which acetylcholine levels are expected to be low or absent.

Cholinesterase and Butyrylcholinesterase in Diabetes Mellitus, Hypertension, Insulin Resistance, Hyperlipidemia and Coronary Heart Disease

It is evident from the preceding discussion that acetylcholinesterase and butyrylcholinesterase are present in various regions of the brain and are increased in the brains of patients with Alzheimer's disease. Furthermore, the activities of these two enzymes seem to be closely associated with the disease activity itself. Thus, higher the activity of acetylcholinesterase and butyrylcholinesterase, more severe the manifestations of Alzheimer's disease and increasing number of cortical and neocortical amyloid-rich neuritic plaques and neurofibrillary tangles [74, 88, 89]. It is interesting to note that changes in the activities of acetylcholinesterase and butyrylcholinesterase have also been reported in other diseases.

Acetylcholinesterase was found to be about an order of magnitude higher in islets of Langerhans than in the exocrine tissue in rat pancreas. This difference in activity was found in rats made diabetic with streptozotocin as well as in the controls [90]. Abbott et al [91] reported that the activity of serum butyrylcholinesterase was significantly elevated in both type 1 (8.10±3.35 units/ml) and type 2 (7.22±1.95 units/ml) diabetes compared with the control subjects (4.23±1.89 units/ml) (P<0.001). In addition, serum butyrylcholinesterase activity correlated with serum fasting triacylglycerol concentration and insulin sensitivity in patients with type 1 and type 2 diabetes. On the other hand, in non-diabetic subjects with butyrylcholinesterase deficiency serum triacylglycerol levels were in the normal range. These results suggested that butyrylcholinesterase might have a role in the altered lipoprotein metabolism in hypertriglyceridemia associated with insulin insensitivity or insulin deficiency in diabetes mellitus [91].

In contrast, streptozotocin diabetes did not affect acetylcholinesterase activity in the retina but increased its activity in the cerebral cortex (100%) and in serum (55%), and decreased it by 30-40% in erythrocytes. The butyrylcholinesterase activity was decreased by 30-50% in retina and hippocampus and to a lesser extent in retinal pigment epithelium from rats treated with streptozotocin for one week. The changes noted in cholinesterase activities were not correlated with the fasting blood glucose concentration. These results suggest that diabetes might influence a specific subset of cells and isoforms of cholinesterases that could lead to alterations associated with diabetes complications [92-94]. It was also reported that the butyrylcholinesterase K variant allele was more common among Type II diabetic subjects than non-diabetic subjects suggesting that the close association of the butyrylcholinesterase gene (3q26) with Type II diabetes could be related to an identified susceptibility locus on chromosome 3q27 but independent of islet function [95]. Since elevated serum butyrylcholinesterase activity is elevated in the diabetic rat, mouse and humans, Dave and Katyare studied the source of the increased level of butyrylcholinesterase and reported that in alloxan-induced diabetic animals both the serum and cardiac butyrylcholinesterase activities were increased 2.2- to 2.8-fold with almost no significant change in the activity of the enzyme after insulin treatment compared with controls [96]. Furthermore, correlation analysis showed that butyrylcholinesterase activity was positively correlated with age, sex, body mass index, hypertension and diabetes, as well as with triglycerides, total cholesterol, low-density lipoprotein cholesterol and apolipoprotein B (Apo B), whereas a step-wise multiple regression analysis revealed that the only risk factors for coronary heart disease that showed independent correlations with butyrylcholinesterase activity were, in descending order of importance, Apo B, triglycerides, and diabetes. These findings reinforce the idea that butyrylcholinesterase activity is associated with lipoprotein synthesis, hypertension, and diabetes [97]. These results emphasize the fact that plasma (serum), red blood cells and leukocyte activities of enzymes butyrylcholinesterase and acetylcholinesterase are elevated in patients with Alzheimer's disease, diabetes mellitus, hypertension, insulin resistance, and hyperlipidemia [88-100]. These results are reinforced by the results of a recent study wherein it was noted that butyrylcholinesterase activity was inversely related to age and was positively associated with serum concentrations of albumin, cholesterol, and triglycerides, and measures of overweight, obesity, and body fat distribution. In multivariate analysis, the associations of enzyme activity with serum cholesterol, triglycerides, and albumin persisted strongly, and paradoxically individuals in the lowest quintile of butyrylcholinesterase activity had significantly higher mortality than those in the highest quintile: all-cause mortality and cardiovascular deaths. The association was attenuated by introduction of serum albumin into the models. These results suggest that low butyrylcholinesterase activity may be a nonspecific risk factor for mortality in the elderly [101]. This indicates that in patients with Alzheimer's disease, diabetes mellitus, hypertension, insulin resistance, and hyperlipidemia the activity of butyrylcholinesterase is not only increased [88-100, 102-104] but also suggests that as and when the activity of the enzyme is low these subjects are at high-risk of death. The exact reason for this increased risk of death in those who have reduced activity of the enzyme butyrylcholinesterase is not clear but it indicates that the reduced activity of the enzyme could be as a result of exhaustion of its stores and/or lowered synthesis. Thus, in the initial stages of the diseases: Alzheimer's disease, diabetes mellitus, hypertension, insulin resistance, and hyperlipidemia the activity of the enzyme butyrylcholinesterase is increased whereas in the terminal stages of the disease or when these diseases are advanced and not easily amenable to treatment butyrylcholinesterase activity is low. These results are imply that the activity of the enzyme butyrylcholinesterase can be used as a marker to predict the prognosis of these diseases.

Alzheimer's Disease as a Low-Grade Systemic Inflammatory Condition

Several studies revealed that Alzheimer's disease is an inflammatory condition. It was reported that plasma and cerebrospinal fluid levels of pro-inflammatory cytokines: interleukin-1 (IL-1) and tumor necrosis factor-α (TNF-α) are increased in patients with Alzheimer's disease and also that of anti-inflammatory cytokine: transforming growth factory-β (TGF-β) [105-107]. The increase in the levels of TGF-β was considered to be a protective host response to immunologically mediated neuronal injury induced by IL-1 and TNF-α. Subsequent studies revealed that systemic injection of IL-1 decreased extracellular acetylcholine in the hippocampus suggesting that increased concentrations of IL-1 in patients with Alzheimer's disease could be responsible for lowered cerebral acetylcholine levels seen in this condition. In addition, IL-1 stimulates the beta-amyloid precursor protein promoter, which is processed out of the larger amyloid precursor protein (APP), which is found in the form of Amyloid plaques in the brains of Alzheimer's diseased patients. Furthermore, receptors of IL-1 are on APP mRNA positive cells and its ability to promote APP gene expression suggests that IL-1 plays an important role in Alzheimer's disease [108, 109]. The involvement of inflammatory process in the pathogenesis of Alzheimer's disease is further supported by the observation that inhibition or neutralizing the actions of TNF-α could be of benefit to these patients [110, 111].

Metabolic Syndrome X, Obesity, Type 2 Diabetes, Insulin Resistance, Hyperlipidemia, Coronary Heart Disease, and Hyperlipidemia are Low-Grade Systemic Inflammatory Conditions

Metabolic syndrome X is characterized by abdominal obesity, atherosclerosis, insulin resistance and hyperinsulinemia, hyperlipidemias, essential hypertension, type 2 diabetes mellitus, and coronary heart disease (CHD). Other features of metabolic syndrome X also include: hyperfibrinogenemia, increased plasminogen activator inhibitor-1 (PAI-1), low tissue plasminogen activator, nephropathy, microalbuminuria, and hyperuricemia. Although the incidence of metabolic syndrome X is assuming epidemic proportions in almost all countries around the globe, the cause(s) for this increasing incidence is not clear.

There is evidence to suggest that low-grade systemic inflammation occurs in metabolic syndrome X [112-114]. Plasma levels of C-reactive protein (CRP), TNF-α, and IL-6, markers of inflammation, are elevated in subjects with obesity, insulin resistance, essential hypertension, type 2 diabetes, and CHD [112-120]. A direct positive correlation exists between BMI (body mass index) and CRP in otherwise healthy children and adults. Higher plasma CRP concentrations is associated with increased risk of CHD, ischemic stroke, peripheral arterial disease, and ischemic heart disease mortality in healthy men and women [113]. Similarly, a strong correlation between elevated CRP levels and cardiovascular risk factors, fibrinogen, and HDL (high-density lipoprotein) cholesterol has also been reported, suggesting that inflammation occurs throughout life and that it participates in the development of atherosclerosis and cardiovascular disease.

IL-6, a pro-inflammatory cytokine stimulates the production of CRP in the liver, and is absolutely required for the induced expression of CRP [113, 121]. This is supported by the observation that higher adipose tissue content of IL-6 is associated with higher serum CRP levels in obese subjects. In overweight and obese subjects, serum levels of TNF-α were also significantly higher compared to lean subjects. Weight reduction or regular exercise decreases serum concentrations of TNF-α. A negative correlation exists between plasma TNF-α and HDL cholesterol, glycosylated hemoglobin, and serum insulin concentrations [reviewed in 113].

Obesity is common in subjects with insulin resistance, type 2 diabetes mellitus, and hypertension. Subjects with elevated plasma CRP levels at baseline testing are at least two times more likely to develop diabetes at 3-4 years of follow-up period [122]. TNF-α secretion is suppressed in younger subjects in response to glucose challenge but not in the older group [123]. Hyperglycemia induces the production of acute phase reactants from the adipose tissue [124], suggesting that glucose is a potent stimulant of inflammatory events especially in elderly compared to young. TNF-α has a role in insulin resistance and type 2 diabetes mellitus. The stimulus for the elevation in the levels of IL-6, TNF-α, and CRP in subjects with type 2 diabetes seems to be hyperglycemia. Esposito et al [125] reported that when plasma glucose levels were acutely raised in control and impaired glucose tolerance (IGT) subjects and maintained at 15 mmol/L for 5 hours while endogenous insulin secretion was blocked with octreotide, in control subjects plasma IL-6, TNF-α, and IL-18 levels rose but were much lower compared to those seen in IGT. In IGT subjects the fasting IL-6 and TNF-α levels were higher than those of control subjects, and the increase in plasma cytokines levels lasted longer compared to control subjects. This increase in plasma cytokine levels was abrogated by simultaneous administration of antioxidant glutathione, suggesting that oxidant stress is involved in increases in circulating cytokines concentrations induced by glucose. Dietary glycemic load is also significantly and positively associated with plasma CRP in healthy middle-aged women [126]. Glucose challenge stimulates generation of reactive oxygen species by leukocytes and decreases vitamin E levels simultaneously. Thus, oxidative stress and pro-inflammatory process could be the underlying molecular events whereby a high intake of carbohydrates increases the risk of insulin resistance and CHD [127]. In addition, high calorie diet rich in fats (especially saturated and trans-fats) or protein stimulate the production of reactive oxygen species [128] by increasing production of IL-6, TNF-α, and IL-18, and CRP. IL-6 and TNF-α activate NADPH oxidase and enhance the generation of reactive oxygen species [129]. These data imply that increased free radical generation in insulin resistance and type 2 diabetes mellitus are due to enhanced production of IL-6, TNF-α, and CRP, which in turn enhance NADPH oxidase activity. Based on these studies, it is clear that consumption of energy dense diets induce a state of oxidative stress by enhancing the production of pro-inflammatory cytokines and free radical generation that are toxic to pancreatic β cells and also produce long-term complications seen in diabetes, hypertension, and CHD.

Essential Hypertension is an Inflammatory Condition

Elevated circulating IL-6 levels in women with hypertension and insulin resistance in men has been described [130]. A significant graded relationship between blood pressure and levels of ICAM-1 (intercellular adhesion molecule-1) and IL-6 was noted [131]. Increase in pulse pressure is associated with elevated CRP among healthy US adults [132]. A direct correlation between plasma CRP levels and advancing age, BMI, systolic blood pressure, HDL, smoking, and hormone replacement therapy was reported in the Women's Health Study [133]. These data indicate that hypertension is associated with low-grade systemic inflammation. We observed that in uncontrolled essential hypertension, elevated plasma lipid peroxides and significantly higher levels of leukocyte superoxide anion and low NO and decreased vitamin E and superoxide dismutase (SOD) in RBC membranes occurred [134]. These abnormalities reverted to normal following control of blood pressure with various anti-hypertensive drugs. Angiotensin II activates leukocyte NADPH oxidase [135] and enhanced superoxide anion generation [134]. Angiotensin converting enzyme (ACE) inhibitors and angiotensin-II receptor blockers enhance plasma adiponectin concentrations and thus, reduce insulin resistance [136]. We observed that β-blockers and calcium antagonists suppressed superoxide anion generation similar to ACE inhibitors [134]. In view of this, it is likely that β-blockers and calcium antagonists augment plasma adiponectin levels similar to ACE inhibitors and angiotensin II receptor blockers. I suggest that anti-hypertensive drugs (except for β-blockers) not only reduce peripheral vascular resistance but also enhance insulin action by augmenting adiponectin secretion. This interaction between anti-hypertensive drugs and adipose tissue needs further evaluation to know whether the former can influence the synthesis and release of leptin, pro- and anti-inflammatory cytokines and energy metabolism [137].

Metabolic Syndrome X is a Low-Grade Systemic Inflammatory Condition

Elevation in the concentrations of pro-inflammatory cytokines, CRP, and free radicals; and decrease in eNO, anti-oxidants, anti-inflammatory cytokines, and adiponectin is common in abdominal obesity, insulin resistance, type 2 diabetes mellitus, hypertension, CHD, and hyperlipidemia [138-140]. This implies that metabolic syndrome X is an inflammatory condition [112]. TNF-α and IL-6 enhance whereas insulin-like growth factor-I (IGF-I) and insulin suppress the activity of 11β-HSD-1 [141]. On the other hand, insulin and IGFs suppress TNF-α and IL-6 and stimulate eNO synthesis and thus, show anti-inflammatory actions [142-146]. Lower concentrations of IGF-I was noted in growth retarded newborn babies [147] and malnourished pregnancies, instances that predispose to the development of type 2 diabetes mellitus, hypertension, and coronary heart disease in adult life. This is supported by the observation in animal studies wherein it was noted that over nutrition leading to catch up growth causes obesity and other features of metabolic syndrome X [148]. This can be attributed to the negative control exerted by IGFs and insulin on MIF (macrophage migration inhibitory factor), TNF-α and IL-6 and 11β-HSD-1. In addition, insulin and possibly, IGFs enhance the production of IL-4 and IL-10, which are potent anti-inflammatory molecules [142, 143]. This suggests that hyperinsulinemia, a marker of insulin resistance, suppresses synthesis of pro-inflammatory cytokines IL-6 and TNF-α and enhances that of anti-inflammatory cytokines IL-4 and IL-10 seen in metabolic syndrome X. In the event hyperinsulinemia fails to restore the balance between pro- and anti-inflammatory cytokines triggered by energy dense diet, low-grade systemic inflammation persists. Both hyperinsulinemia and hyperleptinemia are seen in obese children [149, 150] suggesting that low-grade systemic inflammation and metabolic syndrome X are initiated at an early age.

11β-HSD-1 activity in adipose tissue is regulated by insulin, IGFs, TNF-α, and IL-6. The final expression of 11β-HSD-1 in the abdominal fat depends on the balance between TNF-α and IL-6 and insulin and IGFs [151]. Hence, presence of abdominal obesity is an indication of elevated plasma/tissue levels of CRP, TNF-α, and IL-6, insulin resistance and hyperinsulinemia and decreased levels of IGFs and insulin (qualitative decrease) and increased expression and activity of 11β-HSD-1 in abdominal adipose tissue. TNF-α and IL-6 induce insulin resistance; reduce adiponectin, and eNO synthesis. Elevated concentrations of TNF-α and IL-6 are associated with low plasma HDL and elevated LDL levels, hypertriglyceridemia, hyperleptinemia, and glucose intolerance, abnormalities that are common in subjects with abdominal obesity. These events ultimately lead to metabolic syndrome X.

Psoriasis, Renal Conditions Such as Chronic Renal Failure, End-Stage Renal Disease, Glomerulonephritis Such as Minimal Change Nephropathy, Various Forms of Proliferative Glomerulonephritis, Nephritis Secondary to Underlying Systemic Diseases Such as Collagen Vascular Diseases, Lymphoma and Leukemias, and Cancer are Inflammatory Conditions

It is very well known that conditions such as psoriasis, renal conditions such as chronic renal failure, end-stage renal disease, glomerulonephritis such as minimal change nephropathy, various forms of proliferative glomerulonephritis, nephritis secondary to underlying systemic diseases such as collagen vascular diseases, lymphoma and leukemias, and cancer are all characterized by inflammation since in all these conditions plasma CRP, IL-6, TNF-α, MIF, HMGB1 (high mobility group box 1) and other markers of inflammation are increased. Since it is well accepted that all these conditions are inflammatory conditions, it does not need any further discussion and elaboration.

Acetylcholine is an Anti-Inflammatory Molecule

Acetylcholine (ACh), the natural agonist for the receptors that also bind nicotine. ACh is synthesized in the body from choline and acetylCoA by the enzyme choline acetylase (also referred to as choline acetyltransferase, or CAT). In the laboratory, AChCl (acetylcholine chloride) can be synthesized from trimethylame and beta-chloroethyl acetate.

The chemical formula for AChCl is C7H16ClNO2, for a molecular mass of 181.68. In proper nomenclature, AChCl is 2-(Acetyloxy)-N, N, N-trimethylethanamium chlorode. ACh is also known by the names Acecoline, Arterocolin, Miochol, and Ovisot. The structure of ACh is:

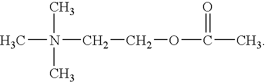

The receptors that recognize ACh are acetylcholine receptors or AChRs. There are two major types (or classes) of acetylcholine receptors in the body, and they are commonly named by the drugs that bind to them: nicotine and muscarine. Muscarinic acetylcholine receptors (mAChRs) can bind muscarine as well as Ach.

Acetylcholine acts on nicotine acetylcholine receptors to open a channel (pore or hole) in the cell's membrane. Nicotinic AChRs are found throughout the body, but they are most concentrated in the nervous system (the brain, the spinal cord, and the rest of the nerve cells in the body) and on the muscles of the body (in vertebrates). The most studied nAChRs are in fact those on the muscles, because those receptors are what cause the muscle to get excited and contract. If a muscle is dissected, and nicotine is applied to it, the muscle will contract. Hence, nicotine acts like ACh at the receptors to activate them, and such substances are called agonists. The opposite type of drug, something that binds to the receptors and does not allow them to be activated is called an antagonist.

When a substance comes into the body that can interfere with ACh binding to muscle nAChRs, that chemical can cause death in a relatively short time. A class of chemicals in snake and other poisonous venoms, called as neurotoxins, do exactly that.

One human neuromuscular disease that is related to nAChRs is myasthenia gravis In the nervous system, the actions of nAChRs are not well characterized. It is known that nicotine is capable of causing addiction. It is also known that nicotine's effects are diverse and at least somewhat dependent on its actions within the nervous system. One complication is that several types of nicotinic receptors are expressed in the nervous system.

Some “subtypes” of nAChRs are expressed in different regions of the brain and peripheral nervous system, but some types of cells express many classes of the receptors. What we do know is that each of these classes is just a little different. Some are more sensitive to nicotine than others. Some activate quickly and then turn off (desensitize) while others stay active as long as the agonist (ACh, nicotine, etc.) is present. These differences are the potential basis for therapies, because hope that there is at least one drug out there or to be designed which can selectively interact with each of these different subtypes.

All known nicotinic receptors do share some common features. They are composed of 5 protein subunits which assemble like barrel staves around a central pore. Currently, we believe that each of these subunits crosses the cell membrane 4 times. Each receptor consists of at least two ligand-binding subunits (called “alpha”) and additional “structural” subunits. When the ligand (ACh or nicotine) binds to the receptor, it causes the receptor complex to twist and open the pore in the center.

The acetylcholine receptor modulates interactions between the nervous system and the immune system. An acetylcholine receptor agonist, nicotine, is now harnessed to dampen inflammation and reduce mortality in a mouse model of sepsis.

The nervous system communicates with the immune system in a bi-directional pathway. Nervous tissues synthesize neuropeptides and cytokines and immune cells and serve as the molecular basis of neural-immune interactions. Neural modulation can have both pro- and anti-inflammatory effects. Unmyelinated sensory C fibers, found in all major organs and tissues, store substance P and other proinflammatory tachykinins and release them in response to bacterial products, tissue injury and other noxious stimuli. Circulating monocytes and tissue macrophages express the neurokinin-1 receptor and serve as an additional source of these proinflammatory peptides.

The cholinergic anti-inflammatory pathway signals through the efferent vagus nerve and is mediated primarily by nicotinic acetylcholine receptors on tissue macrophages—the pathway leads to decreased NF-κB activation, preservation of HMGB1 nuclear localization and decreased production of proinflammatory cytokines. Activation of the sympathetic nervous system also has predominantly anti-inflammatory effects that are mediated through direct nerve to immune cell interaction or through the adrenal neuro-endocrine axis. Interaction of norepinephrine and epinephrine with β-adrenergic receptors on immune cells leads to decreased production of proinflammatory cytokines and increased production of anti-inflammatory cytokines. In the very early stages of acute inflammation, catecholamines may also exert some proinflammatory effects through the α2 adrenoreceptor on macrophages. There is new evidence that activation of afferent vagus nerve fibers by endotoxin or proinflammatory cytokines stimulates hypothalamic-pituitary-adrenal anti-inflammatory responses that lead to anti-inflammatory signals through the efferent vagus nerve, which has been termed the cholinergic anti-inflammatory pathway [152]. This anti-inflammatory pathway is mediated primarily through nicotinic acetylcholine receptors that are expressed on macrophages. Binding of acetylcholine results in reduced nuclear factor (NF-κB) activation, preservation of high mobility group box 1 protein (HMGB1) nuclear localization and reduced production of inflammatory cytokines. Modulation of this axis through direct electrical stimulation of the peripheral vagus nerve during lethal endotoxemia in rats inhibits tumor necrosis factor-α synthesis in the liver, attenuates serum tumor necrosis factor-α levels, and prevents the development of shock [153]. Wang et al. [154] have used nicotine to activate the cholinergic anti-inflammatory pathway and report that this treatment reduced mortality in mice with polymicrobial peritonitis from 84 to 51%—even when administered after the mice appeared clinically ill. The authors provide in vitro evidence that nicotine is associated with a reduction in activation of the proinflammatory transcriptional factor NF-κB in macrophages, an effect that is mediated by the specific nicotinic acetylcholine receptor, α7nAChR.

The efficacy of nicotine may also be explained in part by an inhibitory effect on release of HMGB1, a potentially important late mediator of lethal sepsis that this research group first identified in 1999 [155]. Nicotine treatment was associated with a reduction in serum levels of HMGB1; nicotine also preserved nuclear localization of HMGB1, thereby preventing its release into the extracellular compartment.

Acetylcholinesterase and Butyrylcholinesterase Serve as Markers of Inflammation

Both local and systemic inflammation play a significant role in the pathogenesis of conditions such as insulin resistance, type 2 diabetes mellitus, hypertension, hyperlipidemias, Alzheimer's disease, proliferative diabetic retinopathy; other eye disorders such as macular degeneration, and minimal change nephropathy. In addition, inflammatory events both acute and chronic are at the centre of conditions such as rheumatoid arthritis (RA), systemic lupus erythematosus (SLE), progressive systemic sclerosis (PSS), mixed connective tissue disorder (MCTD), vasculitis; psoriasis, glomerulonephritis of different etiologies including but not limited to proliferative glomerulonephritis, nephritis secondary to underlying systemic diseases such as collagen vascular diseases, lymphoma and leukemias, and central nervous system disorders such as multiple sclerosis. In all these acute, chronic and low-grade systemic inflammatory conditions uncontrolled angiogenic activity also occurs that either precedes or closely follows cell proliferation (for example as seen in proliferative glomerulonephritis and diabetic retinopathy). Our own research showed that in all these conditions the plasma, RBC, leukocyte, platelet, and other tissue (including cerebrospinal fluid) concentrations of acetylcholinesterase and butyrylcholinesterase enzyme activities are increased. Acetylcholine is an anti-inflammatory molecule. Hence, when the concentrations of enzymes acetylcholinesterase and butyrylcholinesterase are increased it will lead to reduced levels of acetylcholine. This leads to absence or a reduction in the anti-inflammatory actions exerted by acetylcholine. Thus, increased plasma, CSF, leukocyte, RBC, platelet, and other tissue concentrations of acetylcholinesterase and butyrylcholinesterase enzymes indirectly reflect reduced concentrations of acetylcholine and so increase in the local and systemic inflammation. Our research also revealed that the activities of the enzymes acetylcholinesterase and butyrylcholinesterase are increased in all the conditions enumerated above even when plasma and CSF and tissue concentrations of CRP, IL-6, TNF-α and other markers of inflammation are not elevated appreciably. Thus, increase in the activities of enzymes acetylcholinesterase and butyrylcholinesterase in the plasma, CSF, RBC, leukocytes, platelets, and other tissues form a reliable, unique and specific marker to detect acute, chronic and low-grade systemic inflammation.

Concept

Both local and systemic inflammation play a significant role in the pathogenesis of conditions such as insulin resistance, type 2 diabetes mellitus, hypertension, hyperlipidemias, Alzheimer's disease, proliferative diabetic retinopathy; other eye disorders such as macular degeneration, and minimal change nephropathy. In addition, inflammatory events both acute and chronic are at the centre of conditions such as rheumatoid arthritis (RA), systemic lupus erythematosus (SLE), progressive systemic, sclerosis (PSS), mixed connective tissue disorder (MCTD), vasculitis; psoriasis, glomerulonephritis of different etiologies including but not limited to proliferative glomerulonephritis, nephritis secondary to underlying systemic diseases such as collagen vascular diseases, lymphoma and leukemias, and central nervous system disorders such as multiple sclerosis. Our own research showed that in all these conditions the plasma, RBC, leukocyte, platelet, and other tissue (including cerebrospinal fluid) concentrations of acetylcholinesterase and butyrylcholinesterase enzyme activities are increased. Acetylcholine is an anti-inflammatory molecule. Hence, when the concentrations of enzymes acetylcholinesterase and butyrylcholinesterase are increased it will lead to reduced levels of acetylcholine. This leads to absence or a reduction in the anti-inflammatory actions exerted by acetylcholine. Thus, increased plasma, CSF, leukocyte, RBC, platelet, and other tissue concentrations of acetylcholinesterase and butyrylcholinesterase enzymes indirectly reflect reduced concentrations of acetylcholine and so increase in the local and systemic inflammation. Our research also revealed that the activities of the enzymes acetylcholinesterase and butyrylcholinesterase are increased in all the conditions enumerated above even when plasma and CSF and tissue concentrations of CRP, IL-6, TNF-α and other markers of inflammation are not elevated appreciably. Thus, increase in the activities of enzymes acetylcholinesterase and butyrylcholinesterase in the plasma, CSF, RBC, leukocytes, platelets, and other tissues form a reliable, unique and specific marker to detect acute, chronic and low-grade systemic inflammation. The activities of acetylcholinesterase and butyrylcholinesterase enzymes is detected or estimated by using simple turbimetric test, or by using ELISA (enzyme linked immunosorbent assay), fluorometric method. radioimmunoassay, spectrophotometric method, wherein specific polyclonal or monoclonal antibodies to enzymes acetylcholinesterase and butyrylcholinesterase enzymes are used and the second antibody conjugated to alkaline phosphatase enzyme or any other suitable enzyme can be used. It is also envisaged in this invention that any other suitable method of detection can be employed to measure the activities of acetylcholinesterase and butyrylcholinesterase enzymes.

### SUMMARY OF THE INVENTION

All the above factors and observations attest to the fact that inflammation plays an important roles in many clinical conditions. In view of the significant role of inflammatory events in various diseases including cancer, several attempts have been made and are being made to develop specific, reliable, and simple tests to detect low-grade systemic inflammation. Success in such attempts is expected to help in the early detection and easy follow up of patients with various diseases in which low-grade systemic inflammation plays a role. But, it should be mentioned here that such attempts to develop specific and simple tests to detect low-grade systemic inflammation have not been very successful.

The present invention specifically teaches the efficacious detection of acetylcholinesterase and butyrylcholinesterase enzymes in various body fluids including but not limited to cerebrospinal fluid (CSF), plasma, serum, and cells such as RBC, leukocytes, platelets, and other biopsy tissues such as synovial biopsy specimens, etc., by using simple turbimetric test, or by using ELISA (enzyme linked immunosorbent assay), fluorometric method. radioimmunoassay, spectrophotometric method, wherein specific polyclonal or monoclonal antibodies to enzymes acetylcholinesterase and butyrylcholinesterase enzymes are used and the second antibody conjugated to alkaline phosphatase enzyme or any other suitable enzyme can be used.

Described hereinafter is a novel test that is simple to do and yet elegant and specific to detect very low levels of acetylcholinesterase and butyrylcholinesterase enzymes are detected even when other conventional tests to detect inflammation are not positive.

The objective of the invention is to outline a simple, elegant, and reliable test for the detection of low amounts of acetylcholinesterase and butyrylcholinesterase enzymes in various tissues, body fluids, and biopsy specimens using a monoclonal and polyclonal antibody (dies) against acetylcholinesterase and butyrylcholinesterase enzymes and detect the activities of these enzymes by various methods including but not limited to turbimetric test, or by using ELISA (enzyme linked immunosorbent assay), fluorometric method. radioimmunoassay, spectrophotometric method. Another objective of the invention is to provide a simple and new method of detecting low-grade systemic inflammation in the form of detecting the activities of enzymes acetylcholinesterase and butyrylcholinesterase even in situations wherein the conventional markers of inflammation such as hs-CRP (high-sensitive CRP), IL-6 TNF-α, MIF, HMGB1, etc., are not detectable even in the presence of diseases enumerated above.

The test system of the invention contains a monoclonal and polyclonal antibody (dies) against acetylcholinesterase and/or butyrylcholinesterase enzymes, their receptor(s), genes of these enzymes, and one or more of secondary antibodies tagged to an enzyme such as alkaline phosphatase or any other suitable enzymes whose activity directly or indirectly gives the quantification of the acetylcholinesterase and/or butyrylcholinesterase enzymes when the test system is employed. The test system proposed can be used in an automated fashion for high throughput detection of many samples, or can be used as a single test or multiple tests as the case may be. The test system can be used daily, weekly, or monthly or at some other appropriate time of interval to detect the presence, progress or regression of the various systemic, local, and/or low-grade systemic inflammatory conditions.

### REFERENCES

[1] Das U N. Is metabolic syndrome X an inflammatory condition? Exp Biol Med 2002; 227: 989-997.
[2] Lau D, Mollanau H, Eiserich J P, et al. Myeloperoxidase mediates neutrophil activation by association with CD11/CD18 integrins. Proc Natl Acad Sci USA 2005; 102: 431-436.
[3] Baldus S, Heitzer T, Eiserich J P, et al. Myeloperoxidase enhances nitric oxide catabolism during myocardial ischemia and reperfusion. Free Radic Biol Med 2004; 37: 902-911.
[4] McDonald D M, Thurston G, Baluk P. Endothelial gaps as sites for plasma leakage in inflammation. Microcirculation 1999; 6: 7.
[5] Ferrara N. Role of vascular endothelial growth factor in physiologic and pathologic angiogenesis: therapeutic implications. Semin Oncol 2002; 29: 10.
[6] Gragoudas E S, Adamis A P, Cunningham Jr E T, Feinsod M, Guyer D R, for the VEGF Inhibition Study in Ocular Neovascularization Clinical trial Group. Pegaptanib for neovascular age-related macular degeneration. N Engl J Med 2004; 351: 2805-2816.
[7] Das U N. Recent advances in the pathobiology of septicemia and septic shock. J Assoc Physicians India 2000; 48:1181-1184.
[8] Muller W A. Leukocyte-endothelial cell interactions in the inflammatory response. Lab Invest 2002; 82: 521.
[9] Kumar V, Abbas A K, Fausto N. Acute and chronic inflammation. In: Robbins and Cotran Pathologic Basis of Disease, Elsevier Saunders, 7^{th} edition, 2005, pp. 47-86.
[10] Johnston B, Butcher E C. Chemokines in rapid leukocyte adhesion triggering and migration. Semin Immunol 2002; 14: 83.
[11] Cicchetti G, Allen P G, Glogauer M. Chemotactic signaling pathways in neutrophils: from receptor to actin assembly. Crit rev Oral Biol Med 2002; 13: 220.
[12] Bucci M, Roviezzo F, Posadas I, et al. Endothelial nitric oxide synthase activation is critical for vascular leakage during acute inflammation in vivo. Proc Natl Acad Sci USA 2005; 102: 904-908.
[13] Padma M, Das U N. Effect of cis-unsaturated fatty acids on cellular oxidant stress in macrophage tumor (AK-5) cells in vitro. Cancer Lett 1996; 109: 63-75.
[14] Das U N. Arachidonic acid as a mediator of some of the actions of phorbolmyristate acetate, a tumor promotor and inducer of differentiation. Prostaglandins Leukot Essen Fatty Acids 1991; 42: 241-244.
[15] Das U N, Padma M. Sangeetha P, et al. Stimulation of free radical generation in human leukocytes by various stimulants including tumor necrosis factor is a calmodulin dependent process. Biochem Biophys Res Commun 1990; 167: 1030-1036.
[16] Serhan C N, Hong S, Gronert K, Colgan S P, Devchand P R, Mirick G, Moussignac R-L. Resolvins: a family of bioactive products of omega-3 fatty acid transformation circuits initiated by aspirin treatment that counter proinflammatory signals. J Exp Med 2002; 196: 1025-1037.
[17] Claria J, Serhan C N. Aspirin triggers previously undescribed bioactive eicosanoids by human endothelial cell-leukocyte interactions. Proc Natl Acad Sci USA 1995; 92: 9475-9479.
[18] Hampton M B, Kettle A J, Winterbourn C C. Inside the neutrophil phagosome: oxidants, myeloperoxidase, and bacterial killing. Blood 1998; 92: 3007.
[19] Reeves E P, Lu H, Jacobs H L, et al. Killing activity of neutrophils is mediated through activation of proteases by K⁺ flux. Nature 2002; 416: 291-297.
[20] Aratani Y, Koyama H, Nyui S, Suzuki K, Kura F, Maeda N. Severe impairment in early host defense against Candida albicans in mice deficient in myeloperoxidase. Infect Immun 1999; 67: 1828-1836.
[21] Aratani Y, Kura F, Watanabe H, et al. Relative contributions of myeloperoxidase and NADPH-oxidase to the early host defense against pulmonary infections with Candida albicans and Aspergillus fumigatus. Med Mycol 2002; 40: 557-563.
[22] Aratani Y, Kura F, Watanabe H, et al. In vivo role of myeloperoxidase for the host defense. Jpn J Infect Dis 2004; 57: S15.
[23] Prescott S M, Zimmerman G A, Stafforini D M, McIntyre T M. Platelet-activating factor and related lipid mediators. Annu Rev Biochem 2000; 69:419-445.
[24] Mantovani A, Sozzani S, Introna M: Endothelial activation by cytokines. Ann NY Acad Sci 1997; 832: 93.
[25] Das U N, Padma M, Sagar P S, et al. Stimulation of free radical generation in human leukocytes by various agents including tumor necrosis factor is a calmodulin dependent process. Biochem Biophys Res Commun 1990; 167: 1030-1036.
[26] Argiles J M, Lopez-Soriano J, Busquets S, Lopez-Soriano F J. Journey from cachexia to obesity by TNF. FASEB J 1997; 11: 743-751.
[27] Das U N. Is metabolic syndrome X an inflammatory condition? Exp Biol Med 2002; 227: 989-997.
[28] Das U N. Is obesity an inflammatory condition? Nutrition 2001; 17: 953-966.
[29] Visser M, Bouter L M, McQuillan G M, et al. Elevated C-reactive protein levels in overweight and obese adults. JAMA 1999; 282: 2131.
[30] Hotamisligil G S. The role of TNF-alpha and TNF receptors in obesity and insulin resistance. J Intern Med 1999; 245: 621.
[31] Pradhan A D, Manson J E, Rifai N, Buring J E, Ridker, P M. C-reactive protein, interleukin-6, and risk of developing type 2 diabetes mellitus. JAMA 2001; 286: 327.
[32] Tracy R P. Inflammation, the metabolic syndrome and cardiovascular risk. Int J Clin Pract Suppl 2003; 134:10-17.
[33] Sattar N, Scherbakova O, Ford I, O'Reilly D S, Stanley A, Forrest E, Macfarlane P W, Packard C J, Cobbe S M, Shepherd J; west of Scotland coronary prevention study. Elevated alanine aminotransferase predicts new-onset type 2 diabetes independently of classical risk factors, metabolic syndrome, and C-reactive protein in the west of Scotland-coronary prevention study. Diabetes 2004; 53: 2855-2860.
[34] Matsumoto K, Sera Y, Abe Y, Ueki Y, Tominaga T, Miyake S. Inflammation and insulin resistance are independently related to all-cause of death and cardiovascular events in Japanese patients with type 2 diabetes mellitus. Atherosclerosis 2003; 169: 317-321.
[35] Rossi D, Zlotnik A. The biology of chemokines and their receptors. Annu Rev Immunol 2000; 18:217-242.
[36] Zlotnik A, Yoshie O. Chemokines: a new classification system and their role in immunity. Immunity 2000; 12:121-127.
[37] Yoshie O, Imai T, Nomiyama H. Chemokines in immunity. Adv Immunol 2001; 78:57-110.
[38] Johnston B, Butcher E C. Chemokines in rapid leukocyte adhesion triggering and migration. Semin Immunol 2002; 14:83-92.
[39] Furchgott R F, Zawadzki J V. The obligatory role of endothelial cells in the relaxation of arterial smooth muscle by acetylcholine. Nature 1980; 288:373-376.
[40] Nathan C. Inducible nitric oxide synthase: what difference does it make? J Clin Invest 1997; 100:2417-2423.
[41] Laroux F S, et al: Role of nitric oxide in inflammation. Acta Physiol Scand 2001; 173:113.
[42] Kumar K V, Das U N. Are free radicals involved in the pathobiology of human essential hypertension? Free Rad Res Commun 1993; 19: 59-66.
[43] Das U N, Krishna Mohan 1, Vijay Kumar K, Sravan Kumar G and Chandra Sekhar C. Beneficial effect of L-arginine in non-insulin dependent diabetes mellitus: A potential role for nitric oxide. Med Sci Res 1993; 21: 669-670.
[44] Mohan K I, Das U N. Oxidant stress, anti-oxidants and nitric oxide in non-insulin dependent diabetes mellitus. Med Sci Res 1997; 25: 55-57.
[45] Mohan K I, Das U N. Effect of L-arginine-nitric oxide system on chemical induced diabetes mellitus. Free Rad Biol Med 1998; 25: 757-765.
[46] Nathan C, Shiloh M U. Reactive oxygen and nitrogen intermediates in the relationship between mammalian hosts and microbial pathogens. Proc Natl Acad Sci USA 2000; 97:8841.
[47] Borregaard N, Cowland J B: Granules of the human neutrophilic polymorphonuclear leukocyte. Blood 1997; 89:3503.
[48] Belaaouaj A: Neutrophil elastase-mediated killing of bacteria: lessons from targeted mutagenesis. Microbes Infect 2002; 4:1259.
[49] Borregaard N, Cowland J B: Granules of the human neutrophilic polymorphonuclear leukocyte. Blood 1997; 89:3503.
[50] Beckman J S, Koppenol W H: Nitric oxide, superoxide, and peroxynitrite: the good, the bad, and the ugly. Am J Physiol 1996; 271:C1424.
[51] Babior B M: Phagocytes and oxidative stress. Am J Med 2003; 109:33.
[52] Das U N. Long-chain polyunsaturated fatty acids interact with nitric oxide, superoxide anion, and transforming growth factory to prevent human essential hypertension. Eur J Clin Nutr 2004; 58: 195-203.
[53] Severini C, et al: The tachykinin peptide family. Pharmacol Rev 2002; 54:285.
[54] Harrison S, Geppetti P. Substance P. Int J Biochem Cell Biol 2001; 33:555.
[55] Richardson J D, Vasko M R: Cellular mechanisms of neurogenic inflammation. J Pharmacol Exp Ther 2002; 302: 839.
[56] Wallen N H, Held C, Rehnqvist N, Hjemdahl P. Elevated serum intercellular adhesion molecule-1 and vascular adhesion molecule-1 among patients with stable angina pectoris who suffer cardiovascular death or non-fatal myocardial infarction. Eur Heart J 1999; 20: 1039-1043.
[57] Mulvihill N T, Foley J B, Murphy R T, Curtin R, Crean P A, Walsh M. Risk stratification in unstable angina and non-Q wave myocardial infarction using soluble cell adhesion molecules. Heart 2001; 85: 623-627.
[58] Ridker P M, Rifai N, Pfeffer M, Sacks F, Lepage S, Braunwald E. Elevation of tumor necrosis factor-alpha and increased risk of recurrent coronary events after myocardial infarction. Circulation 2000; 101: 2149-2153.
[59] Patti G, Di Sciascio G, D'Ambrosio A, Dicuonzo G, Abbate A, Dobrina A. Prognostic value of interleukin-1 receptor antagonist in patients undergoing percutaneous coronary intervention. Am J Cardiol 2002; 89: 372-376.
[60] Biasucci L M, Vitelli A, Liuzzo G, Altamura S, Caligiuri G, Monaco C, Rebuzzi A G, Ciliberto G, Maseri A. Elevated levels of interleukin-6 in unstable angina. Circulation 1996; 94: 874-877.
[61] Biasucci L M, Liuzzo G, Fantuzzi G, Caligiuri G, Rebuzzi A G, Ginnetti F, Dinarello C A, Maseri A. Increasing levels of interleukin (IL)-1Ra and IL-6 during the first 2 days of hospitalization in unstable angina are associated with increased risk of in-hospital coronary events. Circulation 1999; 99: 2079-2084.
[62] Thompson S G, Kienast J, Pyke S D, Haverkate F, van de Loo J C. Hemostatic factors and the risk of myocardial infarction or sudden death in patients with angina pectoris. European Concerted Action on Thrombosis and Disabilities Angina Pectoris Study Group. N Engl J Med 1995; 332: 635-641.
[63] Morrow D A, Rifai N, Antman E M, Weiner D L, McCabe C H, Cannon C P, Braunwald E. Serum amyloid A predicts early mortality in acute coronary syndromes: A TIMI 11A substudy. J Am Coll Cardiol 2000; 35: 358-362.
[64] Johnson B D, Kip K E, Marroquin O C, Ridker P M, Kelsey S F, Shaw L J, Pepine C J, Sharaf B, Bairey Merz C N, Sopko G, et al. Serum amyloid A as a predictor of coronary artery disease and cardiovascular outcome in women: the National Heart, Lung, and Blood Institute-Sponsored Women's Ischemia Syndrome Evaluation (WISE). Circulation 2004; 109: 726-732.
[65] Mallat Z, Corbaz A, Scoazec A, Besnard S, Leseche G, Chvatchko Y, Tedgui A. Expression of interleukin-18 in human atherosclerotic plaques and relation to plaque instability. Circulation 2001; 104: 1598-1603.
[66] Mallat Z, Henry P, Fressonnet R, Alouani S, Scoazec A, Beaufils P, Chvatchko Y, Tedgui A. Increased plasma concentrations of interleukin-18 in acute coronary syndromes. Heart 2002; 88: 467-469.
[67] Baldus S, Heeschen C, Meinertz T, Zeiher A M, Eiserich J P, Munzel T, Simoons M L, Hamm C W; CAPTURE Investigators. Myeloperoxidase serum levels predict risk in patients with acute coronary syndromes. Circulation 2003; 108: 1440-1445.
[68] Brennan M L, Penn M S, Lente F V, et al. Prognostic value of myeloperoxidase in patients with chest pain. N Engl J Med 2003; 349: 1595-1604.
[69] Danesh J, Collins R, Appleby P, Peto R. Association of fibrinogen, C-reactive protein, albumin, or leukocyte count with coronary heart disease: meta-analyses of prospective studies. JAMA 1998; 279: 1477-1482.
[70] Ernst E, Resch K L. Fibrinogen as a cardiovascular risk factor: a meta analysis and review of the literature. Ann Intern Med 1993; 118: 956-963.
[71] Kaplay S S. Acetylcholinesterase and butyrylcholinesterase of developing human brain. Biol Neonate 1976; 28: 65-73.
[72] Jope R S, Walter-Ryan W G, Alarcon R D, Lally K M. Cholinergic processes in blood samples from patients with major psychiatric disorders. Biol Psychiatry 1985; 20: 1258-1266.
[73] Perry E, Perry R, Blessed G, Tomlinson B. Changes in brain cholinesterases in senile dementia of Alzheimer type. Neuropathol Appl Neurobiol 1978; 4: 273-277.
[74] Geula C, Mesulam M M. Cholinesterases and the pathology of Alzheimer disease. Alzheimer Dis Assoc Disord. 1995; 9 Suppl 2: 23-28.
[75] Lahiri D, Farlow M, Sambamurti K, Greig N, Giacobini E, Schneider I. A critical analysis of new molecular targets and strategies for drug developments in Alzheimer's disease. Curr Drug Targets 2003; 4: 97-112.
[76] Soreq H, Seidman S. Acetylcholinesterase—new roles for an old actor. Nat Rev Neurosci. 2001; 2: 294-302.
[77] Taylor P. In The Pharmacological Basis of Therapeutics, eds. Hardman J, Limbird J, Molinoff P, Raddon R, Gilman A: McGraw-Hill, New York, pp. 161-176, 1996.
[78] Yu Q, Holloway H, Utsuki T, Brossi A, Greig N. Synthesis of novel phenserine-based-selective inhibitors of butyrylcholinesterase for Alzheimer's disease. J Med Chem 1999; 42: 1855-1861.
[79] Li B, Stribley J, Ticu A, Xic W, Schopfer L, Hammond P, Brimijoin S, Hinrichs S, Lockridge O. Abundant tissue butyrylcholinesterase and its possible function In the acetylcholinesterase knockout mouse. J Neurochem 2000; 75: 1320-1331.
[80] Darvesh S, Hopkins D, Geula C. Neurobiology of butyrylcholinesterase. Nat Rev Neurobiol 2003; 4: 131-138.
[81] Silver A. The Biology of Cholinesterases, Elsevier/Agricultural Reserach Council Institute, New York, pp. 426-447, 1974.
[82] Mesulam M, Guillozet A, Shaw P, Levey A, Daysen E, Lockridge O. Acetylcholinesterase knockouts establish central cholinergic pathways and can use butyrylcholinesterase to hydrolyze acetylcholine. Neuroscience 2002; 110: 627-639.
[83] Arendt T, Bruckner M, Lange M, Bigl V. Changes in acetylcholinesterase and butyrylcholinesterase in Alzheimer's disease resemble embryonic development-a study of molecular forms. Neurochem Int 1992; 21: 381-396.
[84] Giacobini E. Cholinesterases: new roles in brain function and in Alzheimer's disease. Int J Geriatr Psychiatry. 2003; 18(Suppl 1): S1-5.
[85] Graybiel A, Ragsdale C Jr. Pseudocholinesterase staining in the primary visual pathway of the macaque monkey. Nature 1982; 299: 439-442.
[86] Darvesh S, Grantham D, Hopkins D. Distribution of butyrylcholinesterase in the human amygdala and hippocampal formation. J Comp Neurol 1998; 393: 374-390.
[87] Giacobini E, Spiegel R, Enz A, Veroff A, Cutler N. Inhibition of acetyl- and butyryl-cholinesterase in the cerebrospinal fluid of patients with Alzheimer's disease by rivastigmine: correlation with cognitive benefit. J Neurol Trans 2002; 109: 1053-1055.
[88] Guillozet A, Smiley J, Mash D, Mesulam M. Butyrylcholinesterase in the life cycle of amyloid plaques. Ann Neurol 1997; 42: 909-918.
[89] Greig N H, Utsuki T, Ingram D K, Wang Y, Pepeu G, Scali C, Yu Q S, Mamczarz J, Holloway H W, Giordano T, Chen D, Furukawa K, Sambamurti K, Brossi A, Lahiri D K. Selective butyrylcholinesterase inhibition elevates brain acetylcholine, augments learning and lowers Alzheimer's β-amyloid peptide in rodent. Proc Natl Acad Sci USA 2005; 102: 17213-17218.
[90] Godfrey D A, Matschinsky F M. Enzymes of the cholinergic system in islets of Langerhans. J Histochem Cytochem 1975; 23: 645-651.
[91] Abbott C A, Mackness M I, Kumar S, Olukoga A O, Gordon C, Arrol S, Bhatnagar D, Boulton A J, Durrington P N. Relationship between serum butyrylcholinesterase activity, hypertriglyceridemia and insulin sensitivity in diabetes mellitus. Clin Sci (Lond) 1993; 85′:77-81.
[92] Sanchez-Chavez G, Salceda R. Effect of streptozotocin-induced diabetes on activities of cholinesterases in the rat retina. IUBMB Life 2000; 49: 283-287.
[93] Sanchez-Chavez G, Salceda R. Acetyl- and butyrylcholinesterase molecular forms in normal and streptozotocin-diabetic rat retinal pigment epithelium. Neurochem Int 2001; 39: 209-215.
[94] Sanchez-Chavez G, Salceda R. Acetyl- and butyrylcholinesterase in normal and diabetic rat retina. Neurochem Res 2001; 26: 153-159.
[95] Hashim Y, Shepherd D, Wiltshire S, Holman R R, Levy J C, Clark A, Cull C A. Butyrylcholinesterase K variant on chromosome 3 q is associated with Type II diabetes in white Caucasian subjects. Diabetologia 2001; 44: 2227-2230.
[96] Dave K R, Katyare S S. Effect of alloxan-induced diabetes on serum and cardiac butyrylcholinesterases in the rat. J Endocrinol 2002; 175: 241-250.
[97] Alcantara V M, Chautard-Freire-Maia E A, Scartezini M, Cerci M S, Braun-Prado K, Picheth G. Butyrylcholinesterase activity and risk factors for coronary artery disease. Scand J Clin Lab Invest 2002; 62: 399-404.
[98] Mahmoud F F, Haines D D, Abdul H T, Omu A E, Abu-Donia M B. Butyrylcholinesterase activity in gestational diabetes: correlation with lymphocyte subpopulations in peripheral blood. Am J Reprod Immunol 2006; 56: 185-192.
[99] Sridhar G R, Nirmala G, Apparao A, Madhavi A S, Sreelatha S, Rani J S, Vijayalakshmi P. Serum butyrylcholinesterase in type 2 diabetes mellitus: a biochemical and bioinformatics approach. Lipids Health Dis 2005; 4: 18.
[100] Sreedhar G, Thota H, Allam A R, Suresh babu C, Siva Prasad A, Divakar Ch. Alzheimer's disease and type 2 diabetes mellitus: the cholinesterase connection? Lipids Health Dis 2006; 5: 28.
[101] Calderon-Margalit R, Adler B, Abramson J H, Gofin J, Kark J D. Butyrylcholinesterase activity, cardiovascular risk factors, and mortality in middle-aged and elderly men and women in Jerusalem. Clin Chem 2006; 52: 845-852.
[102] Alcnatara V M, Oliveira L C, Rea R R, Suplicy H L, Chautard-Freire-Maia E A. Butyrylcholinesterase activity and metabolic syndrome in obese patients. Clin Chem Lab Med 2005; 43: 285-288.
[103] Randell E W, Mathews M S, Zhang H, Seraj J S, Sun G. Relationship between serum butyrylcholinesterase and the metabolic syndrome. Clin Chem 2005; 38: 799-805.
[104] Valle A, O'Connor D T, Taylor P, Zhu G, Montgomery G W, Slagboom P E, Martin N G, Whitfield J B. Butyrylcholinesterase: association with the metabolic syndrome and identification of 2 gene loci affecting activity. Clin Chem 2006; 52: 1014-1020.
[105] Cacabelos R, Barquero M, Garcia P, Alvaez X A, Varela de Seijas E. Cerebrospinal fluid interleukin-1 beta (IL-1 beta) in Alzheimer's disease and neurological disorders. Methods Find Exp Clin Pharmacol 1991; 13: 455-458.
[106] Fillit H, Ding W H, Buee L, Kalman J, Altstiel L, Lawlor B, Wolf-Klein G. Elevated circulating tumor necrosis factor levels in Alzheimer's disease. Neurosci Lett 1991; 129: 318-320.
[107] Chao C C, Hu S, Frey W H 2^{nd}, Ala T A, Tourtellotte W W, Peterson P K. Transforming growth factor beta in Alzheimer's disease. Clin Diagn Lab Immunol 1994; 1: 109-110.
[108] Donnelly R J, Friedhoff A J, Beer B, Blume A J, Vitek M P. Interleukin-1 stimulates the beta-amyloid precursor protein promoter. Cell Mol Neurobiol 1990; 10: 485-495.
[109] Blume A J, Vitek M P. Focusing on IL-1-promotion of beta-amyloid precursor protein synthesis as an early event in Alzheimer's disease. Neurobiol Aging 1989; 10: 406-408.
[110] Tobinick E, Gross H, Weinberger A, Cohen H. TNF-alpha modulation for treatment of Alzheimer's disease: a 6-month pilot study. Med Gen Med. 2006; 8: 25.
[111] Rosenberg P B. Cytokine inhibition for treatment of Alzheimer's disease. Med Gen Med. 2006; 8: 24.
[112] Das U N. Is metabolic syndrome X an inflammatory condition? Exp Biol Med 2002; 227: 989-997.
[113]. Ford E S. The metabolic syndrome and C-reactive protein, fibrinogen, and leukocyte count: findings from the Third National Health and Nutrition Examination Survey. Atherosclerosis 2003; 168: 351-358.
[114] Das U N. Obesity, metabolic syndrome X, and inflammation. Nutrition 2002; 18: 430-432.
[115] Albert M A, Glynn R J, Ridker P M. Plasma concentration of C-reactive protein and the calculated Framingham coronary heart disease risk score. Circulation 2003; 108: 161-165.
[116] van der Meer I M, de Maat M P M, Hak A E, et al. C-reactive protein predicts progression of atherosclerosis measured as various sites in the arterial tree. The Rotterdam study. Stroke 2002; 33: 2750-2755.
[117] Luc G, Bard J-M, Juhan-Vague I, et al. C-reactive protein, interleukins-6, and fibrinogen as predictors of coronary heart disease. The PRIME study. Arterioscler Thromb Vasc Biol 2003; 23: 1255-1261.
[118] Ridker P M, Cushman M, Stampfer M J, Tracy R P, Hennekens C H. Inflammation, aspirin, and the risk of cardiovascular disease in apparently healthy men. N Engl J Med 1997; 336: 973-979.
[119] Engstrom G, Hedblad B, Stavenow L, Lind P, Janzon L, Lindgarde F. Inflammation-sensitive plasma proteins are associated with future weight gain. Diabetes 2003; 52: 2097-2101.
[120] Mosca L. C-reactive protein-to screen or not to screen. N Engl J Med 2002; 347: 1615-1617.
[121] Castell J V, Gomez-Lechon M J, David M, Horano T, Kishimoto T, Heinrich P C. Recombinant human interleukins-6 (IL-6/BSF-2/HSF) regulates the synthesis of acute phase proteins in human hepatocytes. FEBS Lett 1988; 232: 347-350.
[122] Barzilay J I, Abraham L, Heckbert S R, Cushman M, Kuller L H, Resnick H E, Tracy R P. The relation of markers of inflammation to the development of glucose disorders in the elderly. Diabetes 2001; 50: 2384-2389.
[123] Kirwan J P, Krishnan R K, Weaver J A, Del Aguila L F, Evans W J. Human aging is associated with altered TNF-α production during hyperglycemia and hyperinsulinemia. Am J Physiol 2001; 281: E1137-E1143.
[124] Lin Y, Rajala M W, Berger J P, Moller D E, Barzilai N, Scherer P E. Hyperglycemia-induced production of acute phase reactants in adipose tissue. J Biol Chem 2001; 276: 42077-42083.
[125] Esposito K, Nappo F, Marfella R, et al. Inflammatory cytokine concentrations are acutely increased by hyperglycemia in humans. Role of oxidative stress. Circulation 2002; 106: 2067-2072.
[126] Liu S, Manson J E, Buring J E, Stampfer M J, Willett W C, Ridker P M. Relation between a diet with a high glycemic load and plasma concentrations of high-sensitivity C-reactive protein in middle-aged women. Am J Clin Nutr 2002; 75: 492-498.
[127] Mohanty P, Hamouda W, Garg R, Aljada A, Ghanim H, Dandona P. Glucose challenge stimulates reactive oxygen species (ROS) generation by leucocytes. J Clin Endocrinol Metab 2000; 85: 2970-2973.
[128] Mohanty P, Ghanim H, Hamouda W, Aljada A, Garg R, Dandona P. Both lipid and protein intakes stimulate increased generation of reactive oxygen species by polymorphonuclear leukocytes and mononuclear cells. Am J Clin Nutr 2002; 75: 767-772.
[129] Fan J, Frey R S, Rahman A, Malik A B. Role of neutrophil NADPH oxidase in the mechanism of tumor necrosis factor-α-induced NF-kB activation and intercellular adhesion molecule-1 expression in endothelial cells. J Biol Chem 2002; 277: 3404-3411.
[130] Fernandez-Real J M, Vayreda M, Richart C, et al. Circulating interleukins 6 levels, blood pressure, and insulin sensitivity in apparently healthy men and women. J Clin Endocrinol Metab 2001; 86: 1154-1159.
[131] Chae C U, Lee R T, Rifai N, Ridker P M. Blood pressure and inflammation in apparently healthy men. Hypertension 2001; 38: 399-403.
[132] Abramson J L, Weintraub W S, Vaccarino V. Association between pulse pressure and C-reactive protein among apparently healthy US adults. Hypertension 2002; 39: 197-202.
[133] Bermudez E A, Rifai N, Buring J, Manson J E, Ridker P M. Interrelationships among circulating interleukins-6, C-reactive protein, and traditional cardiovascular risk factors in women. Arterioscler Thromb Vasc Biol 2002; 22: 1668-1673.
[134] Kumar K V and Das U N. Are free radicals involved in the pathobiology of human essential hypertension? Free Rad Res Commun 1993; 19: 59-66.
[135] Seshiah P N, Weber D S, Rocic P, Valppu L, Taniyama Y, Griendling K K. Angiotensin II stimulation of NAD(P)H oxidase activity. Circulation Res 2002; 91: 406-413.
[136] Furuhashi M, Ura N, Higashiura K, et al. Blockade of the rennin-angiotensin system increases adiponectin concentrations in patients with essential hypertension. Hypertension 2003; 42: 76-81.
[137] Das U N. Long-chain polyunsaturated fatty acids interact with nitric oxide, superoxide anion, and transforming growth factor-β to prevent human essential hypertension. Eur J Clin Nutr 2004; 58: 195-203.
[138] Jovinge S, Hamsten A, Tomvall P, et al. Evidence for a role of tumor necrosis factor alpha in disturbances of triglycerides and glucose metabolism predisposing to coronary heart disease. Metabolism 1998; 47: 113-118.
[139] Chu N-F, Spiegelman D, Rifai N, et al. Glycemic status and soluble tumor necrosis factor receptor levels in relation to plasma leptin concentrations among normal weight and overweight US men. Int J Obes 2000; 24: 1085-1092.
[140] Das U N. Nutritional factors in the pathobiology of human essential hypertension. Nutrition 2001; 17: 337-346.
[141] Tomilinson J W, Moore J, Cooper M S, et al. Regulation of expression of 11-hydroxysteroid dehydrogenase type 1 in adipose tissue: Tissue-specific induction by cytokines. Endocrinology 2001; 142: 1982-1989.
[142] Das U N. Is insulin an anti-inflammatory molecule? Nutrition 2001; 17: 409-413.
[143] Phipps K, Barker D J, Hales C N, et al. Fetal growth and impaired glucose tolerance in men and women. Diabetologia 1993; 36: 225-228.
[144] Jeschke M G, Einspanier R, Klein D, et al. Insulin attenuates the systemic inflammatory response. Mol Med 2002; 8: 443-450.
[145] Gao F, Gao E, Yue T-L, et al. Nitric oxide mediates the anti-apoptotic effect of insulin in myocardial ischemia-reperfusion. The roles of PI3-kinase, Akt, and endothelial nitric oxide synthase phosphorylation. Circulation 2002; 105: 1497-1502.
[146] Dandona P, Aljada A, Mohanty P, et al. Insulin inhibits intranuclear factor kappa B and stimulates IkappaB in mononuclear cells in obese subjects: evidence for an antiinflammatory effect. J Clin Endocrinol Metab 2001; 86: 3257-3265.
[147] Abuzzahab M J, Schneider A, Goddard A, Grigorescu F, Lautier C, Keller E, Kiess W, Klammat J, Kratzsch J, Osgood D, Pfaffle R, Raile K, Seidel B, Smith R J, Chernausek S D, for the Intrauterine Growth Retardation (IGR) Study Group. IGF-I receptor mutations resulting in intrauterine and postnatal growth retardation. New Engl J Med 2003; 349: 2211-2222.
[148] Ozanne S E, Hales C N. Lifespan: Catch-up growth and obesity in male mice. Nature 2004; 427: 411-412.
[149] Whincup P H, Gilg J A, Papacosta O, et al. Early evidence of ethnic differences in cardiovascular risk: cross sectional comparison of British South Asian and white children. BMJ 2002; 324: 1-6.
[150] Kalhan R, Puthawala K, Agarwal S, Amini S B, Kalhan S C. Altered lipid profile, leptin, insulin, and anthropometry in offspring of South Asian immigrants in the United States. Metabolism 2001; 50: 1197-1202.
[151] Das U N. Pathobiology of metabolic syndrome X in obese and non-obese South Asian Indians Further discussion and some suggestions. Nutrition 2003; 19: 560-562.
[152] Pavlov, V. A. & Tracey, K. J. Cell Mol. Life Sci. 2004; 61: 2322-2331.
[153] Borovikova, L. V. et al. Nature 2000; 405: 458-462.
[154] Wang, H. et al. Nature Med 2004; 10: 1216-1221.
[155] Wang, H. et al. Science 1999; 285: 248-251.

1. A method of detecting, diagnosing and prognosticating inflammatory conditions by measuring an increase in the activity of acetylcholinesterase and butyrylcholinesterase enzymes.

2. A method as in claim 1, wherein the activity of acetylcholinesterase and butyrylcholinesterase is measured in the plasma or tissue(s).

3. A method wherein a decrease in the activity of plasma and/or tissue acetylcholinesterase and butyrylcholinesterase is used as a marker of response to treatment given.
